(19) United States
(12) Patent Application Publication
(10) Pub. No.: US 2003/0150017 A1
(43) Pub. Date: Aug. 7, 2003

(54) Method for facilitating pathogen resistance

(76) Inventors: Jose Ramon Botella Mesa, Kenmore (AU); Michael Wayne Graham, Jindalee (AU); David James Fairbairn, Chapel Hill (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/291,253

(22) Filed: Nov. 7, 2002

Related U.S. Application Data
(60) Provisional application No. 60/341,404, filed on Dec. 14, 2001.

(30) Foreign Application Priority Data
Nov. 7, 2001 (AU) PR 8706
Nov. 12, 2001 (AU) PR 8802

(51) Int. Cl. A01H 5/00; C12N 15/82

(52) U.S. Cl. 800/279; 435/468; 800/317.3

(57) ABSTRACT

Methods are provided for the genetic control of pathogen infestation in host organisms such as plants, vertebrate animals and fungi. Such methods utilize the host as a delivery system for the delivery of genetic agents, preferably in the form of RNA molecules, to a pathogen, which agents cause directly or indirectly an impairment in the ability of the pathogen to maintain itself, grow or otherwise infest a host plant, vertebrate animal or fungus. Also provided are DNA constructs and novel nematode nucleotide sequences for use in same, that facilitate pathogen resistance when expressed in a genetically-modified host. Such constructs direct the expression of RNA molecules substantially homologous and/or complementary to an RNA molecule encoded by a nucleotide sequence within the genome of a pathogen and/or of the cells of a host to effect down-regulation of the nucleotide sequence. Particular hosts contemplated are plants, such as pineapple plants, and particular pathogens are nematodes.

### RELATED U.S. APPLICATION

The present application claims priority under 35 U.S.C. §119(e) from U.S. Provisional Application No. 60/341,404, filed Dec. 14, 2001.

### FIELD OF THE INVENTION

THIS INVENTION relates generally to the genetic control of pathogen infestation in host organisms such as plants, vertebrate animals and fungi. More particularly, the present invention contemplates the delivery of genetic agents to a pathogen which agents cause directly or indirectly an impairment in the ability of the pathogen to maintain itself, grow or otherwise infest a host plant, vertebrate animal or fungus. In a particular form, the present invention provides a genetically modified plant, vertebrate animal or fungal host which comprises properties which facilitate a reduction in the ability for a pathogen to maintain itself, grow or otherwise infest the host. Consequently, the present invention contemplates the induction or facilitation of resistance or at least increased tolerance of a plant, vertebrate animal or fungus to infection by a pathogen. The present invention provides DNA constructs and novel nematode nucleotide sequences for use in same, the expression of which in a cell or when applied to cells or tissue of a plant, vertebrate animal or fungal host, results in the down-regulation of a nucleotide sequence in the pathogen, thereby causing a deleterious effect on the maintenance, viability and/or infectivity of the pathogen. The present invention is further directed to the expression of a nucleotide sequence transcribable to an RNA sequence which is substantially homologous and/or complementary to an RNA molecule comprising an RNA sequence encoded by a nucleotide sequence within the genome of a pathogen to effect down-regulation of the nucleotide sequence. The down-regulated nucleotide sequence in the pathogen results in a deleterious effect on the maintenance, viability and/or infectivity of the pathogen. The constructs and nucleotide sequences of the present invention may be useful in controlling, ameliorating or otherwise modulating infestation by a range of pathogens in plants, vertebrate animals and/or fungi. Plants and other organisms displaying resistance and/or enhanced tolerance to infestation through the methods of the present invention are also encompassed.

### BACKGROUND OF THE INVENTION

The increasing sophistication of recombinant nucleic acid technology is greatly facilitating research and development in a range of biological industries. This is particularly evident in relation to the agricultural and horticultural industries. A greater understanding of the mechanisms, underlying a number of genetic events permits the exploitation of these mechanisms to create more efficacious genetic agents to alter the properties of plants and animals.

One particularly important area concerns protecting plant crops from pathogens. One pathogen which has a devastating impact on crops is the roundworm or nematode. The roundworm also infects animal tissue and can cause disease in, for example, the animal industry. Nematodes also infect fungi and, hence, the emerging industry of using fungi as biofactories faces production loss if the fungi are infected with nematodes. Consequently, the ability to control nematode pests is of importance to the horticultural and agricultural industries.

Phyla Nematoda and Nemathelminthes, within Kingdom Animalia, consist completely of roundworms. Roundworms are cylindrical, bilaterally symmetrical worm-like organisms. They are surrounded by a cuticle, a tough, flexible non-cellular outer layer that protects the nematode from drying or being crushed. Parasitic roundworms live within the tissues of plants and the body fluid or tissues of other animals. They are the sources of diseases such as trichinosis, pinworms, filariasis (elephantitis) and onchoceriasis (river blindness). Free-living roundworms are more abundant than parasitic roundworms and are just as harmful. They destroy plant roots, causing the entire plant to die, and can deprive an animal of the necessary nutrients it needs to survive. Free-living roundworms, unlike the parasites, are ubiquitious in nature and many of them feed off rotting organic matter. It has been reported, for example, that around 90,000 individual nematodes live in a single rotting apple.

Even when symptom-free, parasitic nematode infections are harmful to the host plant or animal for a number of reasons, e.g. they deprive the host of food, injure organs or obstruct ducts or vascular tissues, may elaborate substances toxic to the host, and provide a port of entry for other organisms. In other cases, the host may be a species raised for food and the parasite may be transmitted upon eating to infect the ingesting animal. It is highly desirable, therefore, to eliminate such parasites as soon as they are discovered.

Parasitism of plants by nematode infestation cause millions of dollars of damage each year to turf grasses, ornamental plants and food crops. Over 20% of the annual yield losses of major crops in the world occur from plant-parasitic nematodes: monetary losses due to nematode infestation is estimated to be U.S.$100 billion worldwide. There is a correspondingly large market for nemacides, or agents which kill nematodes and suppress nematode infestation. Efforts to eliminate or minimize damage caused by nematodes in agricultural settings have typically involved the use of soil fumigation. Such fumigation materials can be highly toxic and may create an environmental hazard.

In plant species, most of the economic damage is caused by the sedentary endoparasitic nematodes of the family Heteroderidae. This family is divided into the cyst nematodes (Heterodera and Globodera) and the root-knot nematodes (Meloidogyne). The root-knot nematodes, so-called for the root galls (root knots) which form on many hosts, infect thousands of plant species and cause severe losses in yield of many crops (vegetables and fruits) as well as flowers and other ornamentals. Symptoms of diseased plants include stunting, wilting and susceptibility to other diseases. Members of the Meloidogyne and Rotylenchulus have particularly devastating effects on many crop species including Ananas comosus and related pineapple species.

Chemical treatment of the soil is one of the most promising means to control plant-parasitic nematodes. Methyl bromide, organophosphates and carbamates are widely used nematocides, which unfortunately are highly hazardous to the environment. Organophosphates and carbamates paralyze the nematode by inhibiting acetylcholinesterase enzyme activity which is essential for neural activity. Although the use of nematocides is effective in reducing the population level of the nematode, nematocide use is both uneconomical and potentially environmentally unsound as a control measure in plant production. Various non-fumigant chemicals have also been used but these too create serious environmental problems and can be highly toxic to humans and animals. Methods such as crop rotationprovide a means of nematode control. However, rotation with a non-susceptible crop for at least two years is required before crop loss is reduced.

Roundworms which infect animals such as mammals, include the hookworm (e.g. Necator americanus and Ancylostoma duodenale), roundworm (e.g. the common roundworm Ascaris lumbricoides), whipworm (e.g. Trichuris trichiura) and the pinworm or threadworm (e.g. Enterobius vermicularus) as well as Stroncyloides stercoralis, Trichinella spiralis (infection in man and pigs) and the filarial worm Wuchereria bancrofti. Other important roundworm parasites include Ancylostoma caninum (infections of man), Stroncylus vulgaris (infections of horses), Trichostrongylus colubriformis (infections of sheep), Haemonchus contortus (infections of sheep and goats), Ostertagia ostertagi (infections of cattle), Ascaris suum (infections in pigs), Toxascaris leonia or Uncinaria stenocephala (infections of dogs), Toxocara spp (circulatory infections of man) and Dirofilaria immitis (circulatory infections of cats and dogs).

In animals including mammals such as humans, parasitic nematode or helminth infection, are typically treated by chemical drugs. Anthelminthic chemicals are used in order to keep such infections under control. Untreated, nematode infestations may result in anaemia, diarrhoea, dehydration, loss of appetite and even death in animals and humans. Treatment requires the use of chemical drugs for the control of nematode infestation. Drugs need to be frequently administered. For example, dogs susceptible to heartworm are typically treated monthly. Repeated administration of drugs can lead to the development of resistant nematode strains that no longer respond to treatment. Furthermore, many of the chemical drugs cause harmful side effects in the animals being treated and as larger doses become required due to the build up of resistance, the side effects become even greater. Moreover, a number of drugs only treat symptoms of a parasitic disease but are unable to prevent infection by the parasitic helminth.

### OBJECT OF THE INVENTION

In the light of such substantial economic losses and the additional expense and danger associated with treating crops and animals with nematode infection, the present inventors have identified a need to develop a method to control not only nematode infection but also infection by other pathogens such as insects.

Ideally, the method would be useful in providing resistance to pathogen infestation across the entire range of parasites including parasites which infect plants, vertebrate animals and fungi. Insect pests cause billions of dollars in losses every year and are difficult to control requiring large amounts of insecticides that are highly toxic to other non-destructive “friendly” insects. In addition, many of the pesticides used are non-biodegradable and accumulate in waterways. Helicoverpa armigera is a serious caterpillar pest worldwide attacking several important crops such as lettuce, potato, sunflower, sorghum, soybean and alfalfa. In Australia, H. armigera is especially damaging to cotton, tomato and maize crops. The species easily develop resistance to many insecticides and special combinations of chemicals are used to control it.

Additionally, the method would obviate the need for use of extremely dangerous chemicals.

It is therefore an object of the invention to exploit mechanisms underlying post-transcriptional gene silencing events and other similar mechanisms to facilitate pathogen resistance in hosts such as plants, vertebrate animals and fungi.

### SUMMARY OF THE INVENTION

The present invention relates generally to a genetic agent delivery system to induce gene silencing in pathogens of hosts including, but not limited to, plants, vertebrate animals and fungi. The term “silencing” in this context includes substantial down-regulation of expression to basal levels as well as partial down-regulation to below “normal” levels. A “vertebrate animal” includes a human, livestock animal, companion animal, laboratory test animal as well as avian species. The reduction in expression of a particular pathogen gene results directly or indirectly in a reduced ability of the pathogen to grow, maintain itself or continue infecting or infesting the plant, vertebrate animal or fungal host. In still another embodiment, non-pathogenic including attenuated strains of microorganisms are engineered to express genetic material to produce RNA molecules comprising RNA sequences homologous or complementary to RNA sequences in cells of a pathogen. The microorganisms are an example of a biological matrix. Exposure of the pathogen to the host results in ingestion of the microorganisms leading to down-regulation of expression of target pathogen genes mediated directly or indirectly by the RNA molecules or fragments or derivatives thereof. In still yet another embodiment, the RNA molecules themselves are encapsulated in a synthetic matrix such as a polymer and applied to the surface of a host. Again, ingestion of host cells, by a pathogen permits delivery of the RNA molecules to the pathogen and results in down-regulation of a target gene in the host.

Preferred pathogens of the present invention are nematodes and insects. The most preferred pathogen is a nematode.

Preferred hosts of the present invention are plants.

In one aspect, the invention provides a method for facilitating host resistance to at least one pathogen, said method including the step of generating a host which comprises one or more nucleotide sequences that is/are transcribable to one or more respective RNA molecules and that is/are substantially homologous and/or complementary to one or more respective nucleotide sequences of the genome of said at least one pathogen, such that upon exposure of said at least one pathogen to said host, there is down-regulation of expression of at least one of said respective nucleotide sequences of the genome of said at least one pathogen which thereby facilitates host resistance to said pathogen.

In one form of this aspect, one or more cells of the host comprise the one or more respective RNA molecules transcribed from said one or more nucleotide sequences. Preferably, ingestion by said at least one pathogen of said one or more respective RNA molecules present in the host cells results in down-regulation of expression of at least one of said respective nucleotide sequences encoded by the genome(s) of said at least one pathogen which thereby facilitates host resistance to said pathogen.

In another form of this aspect, the host comprises one or more substantially non-pathogenic microorganisms associated therewith that comprise the one or more respective RNA molecules transcribed from said one or more nucleotide sequences. Preferably, ingestion of said substantially non-pathogenic microorganisms by said at least one pathogen results in down-regulation of expression of at least one of said respective nucleotide sequences encoded by the genome(s) of said at least pathogen which thereby facilitates host resistance to said pathogen.

In another aspect, the invention provides a method for facilitating host resistance including the step of encapsulating in a synthetic matrix one or more RNA molecules comprising RNA sequences which is/are substantially homologous and/or complementary to an RNA molecule comprising an RNA sequence encoded by the genome of at least one pathogen such that upon exposure to said synthetic matrix when associated with said host, there is down-regulation of expression of said at least one pathogen nucleotide sequence which thereby facilitates host resistance to said at least one pathogen..

According to the aforementioned aspects of the invention, preferably said host is a plant, vertebrate animal or fungal host.

More preferably, the host is a plant.

It will be appreciated by the skilled person that the method of the invention facilitates resistance of said host to said pathogen, or otherwise enhances tolerance of said host to pathogen infection and/or infestation.

The invention also provides a genetic construct for facilitating host resistance to a pathogen.

In a particular embodiment, said genetic construct comprises one or more nucleotide sequences that is/are transcribable to an RNA molecule and that is/are substantially homologous and/or complementary to one or more nucleotide sequences encoded by the genome of said pathogen, such that upon exposure of said pathogen to one or more RNA molecules transcribed from said one or more nucleotide sequences, there is down-regulation of expression of at least one of said respective nucleotide sequences of the genome of said pathogen.

Preferably, said genetic construct comprises one or more nucleotide sequences selected from the group consisting of a those set forth in SEQ ID NO:1; SEQ ID NO;2; SEQ ID NO:3; SEQ ID NO:4; SEQ ID NO:5; SEQ ID NO:6; SEQ ID NO:7; and SEQ ID NO:8.

More preferably, said genetic construct comprises one or more nucleotide sequences selected from the group consisting of a those set forth in SEQ ID NO:1; SEQ ID NO;2 and SEQ ID NO:3.

In one particular embodiment, said genetic construct is pUQC477 or pUQC136.

In a further aspect, the invention provides a genetically-modified host organism, such as a plant, vertebrate animal and fungus, which is resistant to infection and/or infestation by a pathogen.

In particular embodiments, the genetically-modified host is a pineapple plant or a tobacco plant.

In a still further aspect, the invention provides an isolated nucleic acid comprising a sequence of nucleotides selected from the group consisting of those set forth in SEQ ID NO:1; SEQ ID NO;2; SEQ ID NO:3; SEQ ID NO:4; SEQ ID NO:5; SEQ ID NO:6; SEQ ID NO:7; and SEQ ID NO:8.

The invention also extends to homologues, orthologues and fragments of the isolated nucleic acids of this aspect.

Also according to this aspect there is provided a chimeric gene comprising an isolated nucleic acid of the invention and another nucleotide sequence such as an operably linked promoter, a spacer, an intron or expression modulatory sequence (EMS), for example.

It will also be appreciated that the invention provides an isolated protein encoded by the aforementioned isolated nucleic acids.

In a particular embodiment, said isolated protein comprises an amino acid sequence selected from the group consisting of those set forth in SEQ ID NO:9; SEQ ID NO:10; SEQ ID NO:11; SEQ ID NO:12; SEQ ID NO:13; SEQ ID NO:14; SEQ ID NO:15; and SEQ ID NO:16.

Throughout this specification, the standard single-letter amino acid and nucleotide sequence code is used.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

### BRIEF DESCRIPTION OF THE FIGURES

FIG. 1 provides nucleotide sequences of Meloidogynes genes or gene fragments (upper panels) and encoded amino acid sequences (lower panels). A: M. javanica mex1 full length nucleotide sequence (SEQ ID NO:1) and amino acid sequence (SEQ ID NO:9) encoded by bases 47-805; B: M. incognita skn1 partial nucleotide sequence (SEQ ID NO:2) and amino acid sequence (SEQ ID NO:10) encoded by bases 2 to 883; C: M. javanica skn1 partial nucleotide sequence (SEQ ID NO:3) and encoded amino acid sequence (SEQ ID NO:11); D: M. incognita mei 1 partial nucleotide sequence (SEQ ID NO:4) and amino acid sequence encoded by bases 2 to 886; E: M. incognita gbp1 full length nucleotide sequence (SEQ ID NO:5) and amino acid sequence (SEQ ID NO:13) encoded by bases 31 to 990; F: M. incognita dif1 partial nucleotide sequence (SEQ ID NO:6) and amino acid sequence (SEQ ID NO: 14) encoded by bases 39 to 770; G: M. incognita rba2 partial nucleotide sequence (SEQ ID NO:7) and amino acid sequence (SEQ ID NO:15) encoded by bases 2 to 652; H: M. incognita plk1 full length sequence (SEQ ID NO:8) and amino acid sequence (SEQ ID NO:16) encoded by bases 3 to 1052.

FIG. 2 illustrates an agarose gel displaying the products of RT-PCR analysis of M. javanica from infected tomato.

FIG. 3 is a diagrammatic representation of four DNA constructs for inducing resistance in plants, vertebrate animal cells and fungi to pathogen infestation or infection. Ter, terminator; EMS, expression modulating sequences; (diagonal cross-hatching) nucleotide sequence encoding an RNA molecule having an RNA sequence homologous or complementary to an RNA sequence in a pathogen cell.

FIG. 4 is a diagrammatic representation of a hybrid DNA construct comprising multiple pathogen genes or gene fragments (genes 1,2,3,4). This construct is useful for targeting two or more pathogens or to reduce the likelihood of resistance by targeting multiple genes of a single pathogen.

FIG. 5 is a schematic representation of pHannibal plasmid vector used for production of genetic constructs. This vector is also described in Internatonal Application PCT/IB99/00606.

FIG. 6 A-F illustrates various genetic constructs and intermediates used in their construction.

FIG. 7 shows the results of PCR analysis of tobacco genomic DNA.

### DETAILED DESCRIPTION OF THE INVENTION

The present invention provides a means for enabling a host organism, preferably a plant, vertebrate animal or fungus, to exhibit increased tolerance to pathogenic infection. All such plants, vertebrate animals and fungi are referred to herein a “hosts” or as comprising “host cells”. Owing to the enormous losses caused in world-wide agriculture by pathogens, the development of methods for ameliorating their effects is highly desired. The present invention is predicated in part on the delivery of genetic agents to pathogens through their exposure to a host, such as by ingestion of host cells or the contents of said cells. The present invention alternatively provides exposure of a pathogen to these genetic agents encapsulated in biological or synthetic membranous or polymeric matrices and applied to the surface of a host. Typically, ingestion of these biological or synthetic matrices by a pathogen or the infection of the pathogen by same, permits delivery of the genetic agents. The present invention provides, therefore, a genetic agent delivery system. The genetic agents of the present invention induce inter alia directly or indirectly post-transcriptional gene silencing events of target genes in a pathogen such as a nematode. Down-regulation of expression of the target gene prevents or at least retards pathogen growth, development and/or reproduction.

In one form, the genetic agents are single-, double-stranded or partially double-stranded RNA molecules. In another form, the genetic agents are RNAi or other nucleotide sequence-specific ribonuclease-comprising complexes. In yet another form, the genetic agents are DNA constructs which encode RNA molecules. The present invention contemplates, therefore, inter alia genetic agents, genetically modified plants, vertebrate animals and fungi and methods of inducing or otherwise facilitating resistance or reduced susceptibility of a host to a pathogen.

In this context, it will be understood by that the present invention contemplates the following:

(i) delivery of a single pathogen nucleotide sequence to a host;

(ii) delivery of a plurality of nucleotide sequences to a host, said nucleotide sequences derived or obtained from different pathogens to thereby provide resistance to a plurality of pathogens;

(iii) delivery of a single nucleotide sequence to a host, wherein the nucleotide sequence is a conserved region of orthologous nucleotide sequences of different pathogens, to thereby provide resistance to a plurality of pathogens; and

(iv) delivery of a single pathogen nucleotide sequence to a host, wherein the nucleotide sequence is highly specific to that particular pathogen.

An example of (ii) could be delivery of M. javanica and M.incognita nucleotide sequences such as described herein.

An example of (iii) could be delivery of a conserved region of M. javanica and M.incognita skn1 nucleotide sequences, which sequences are highly conserved as described herein.

An example of (iv) could be delivery of a nucleotide sequence corresponding to a relatively non-conserved 5′ or 3′UTR region of a pathogen gene.

Accordingly, one embodiment of the present invention provides a method for facilitating resistance or otherwise enhancing tolerance of a plant, vertebrate animal or fungal host to infection or infestation by a pathogen, said method comprising generating a host which comprises one or more nucleotide sequences transcribable to an RNA molecule comprising an RNA sequence which is substantially homologous and/or complementary to an RNA molecule comprising an RNA sequence encoded by a nucleotide sequence within the genome of said pathogen, such that upon ingestion by said pathogen of said RNA molecule or a fragment or derivative thereof present in host cells, there is down-regulation of expression of said pathogen nucleotide sequence which has a deleterious effect on the maintenance, viability and/or infectivity of said pathogen.

In a related embodiment, there is provided a method for facilitating resistance or otherwise enhancing tolerance of a plant, vertebrate animal or fungal host to infection or infestation by a pathogen, said method comprising generating a substantially non-pathogenic microorganism which comprises one or more nucleotide sequences transcribable to an RNA molecule comprising an RNA sequence which is substantially homologous and/or complementary to an RNA molecule comprising an RNA sequence encoded by a nucleotide sequence within the genome of said pathogen, such that upon ingestion by said pathogen of said RNA molecule or a fragment or derivative thereof present in microbial cells applied to a surface portion of said host, there is down-regulation of expression of said pathogen nucleotide sequence which has a deleterious effect on the maintenance, viability and/or infectivity of said pathogen.

Still another related embodiment of the present invention is directed to a method for facilitating resistance or otherwise enhancing tolerance of a plant, vertebrate animal or fungal host to infection or infestation by a pathogen, said method comprising encapsulating in a synthetic matrix one or more RNA molecules comprising RNA sequences which is/are substantially homologous and/or complementary to an RNA molecule comprising an RNA sequence encoded by a nucleotide sequence within the genome of said pathogen such that upon ingestion of said pathogen when the synthetic matrix is applied to a surface region on said host, there is down-regulation of expression of said pathogen nucleotide sequence which has a deleterious effect on the maintenance, viability and/or infectivity of said pathogen.

According to the first-mentioned aspect, generating the genetically modified host plant, vertebrate animal, fungus or microorganism generally means introducing a double-stranded DNA (dsDNA) genetic construct into cells derived from a particular host and then either regenerating a host plant, animal or fungus therefrom or multiplying the non-pathogenic microorganism. The regenerated or multiple host or progeny thereof which contain the dsDNA construct produce in all, or select, cells the RNA molecule which comprises RNA sequences which are substantially homologous and/or complementary to an RNA molecule comprising an RNA sequence encoded by a nucleotide sequence within the genome of the pathogen. In relation to plants, it is preferable that the RNA molecules are at least produced in giant cells as certain pathogens such as nematodes infect or infest via giant cells. Accordingly, the dsDNA construct may have tissue or developmentally regulated promoters to ensure production of the RNA molecules in the appropriate tissues. With respect to the third of the afore-mentioned aspects, RNA molecules are generated in large amounts and then admixed within the synthetic matrix.

The term “dsDNA construct” means a double-stranded DNA construct and may also be regarded inter alia as a recombinant molecule, a genetic agent, a genetic molecule or a chimeric genetic construct. A chimeric genetic construct of the present invention may comprise, for example, nucleotide sequences encoding one or more antisense transcripts, one or more sense transcripts, one or more of each of the afore-mentioned, wherein all or part of a transcript therefrom is homologous to all or part of an RNA molecule comprising an RNA sequence encoded by a nucleotide sequence within the genome of a pathogen. The latter “nucleotide sequence” is generally a DNA sequence such as a gene sequence but may also be an RNA sequence operably linked to an RNA promoter.

Accordingly, another embodiment of the present invention contemplates a method for facilitating resistance or otherwise enhancing tolerance of a plant, vertebrate animal or fungus host to infection by a pathogen, said method comprising generating a host which comprises one or more nucleotide sequences capable of directing synthesis of an RNA molecule, said nucleotide sequence selected from the list comprising:

(i) a nucleotide sequence transcribable to an RNA molecule comprising an RNA sequence which is substantially homologous to an RNA sequence encoded by a nucleotide sequence within the genome of said pathogen;

(ii) a reverse complement of the nucleotide sequence of (i);

(iii) a combination of the nucleotide sequences of (i) and (ii),

(iv) multiple copies of nucleotide sequences of (i), (ii) or (iii), optionally separated by a spacer sequence;

(v) a combination of the nucleotide sequences of (i) and (ii), wherein the nucleotide sequence of (ii) represents an inverted repeat of the nucleotide sequence of (i), separated by a spacer sequence; and

(vi) a combination as described in (v), wherein the spacer sequence comprises an intron sequence spliceable from said combination;

wherein upon ingestion by said pathogen of said RNA molecule or fragment or derivative thereof present in host cells, there is down-regulation of expression of said pathogen nucleotide sequence within the genome of said pathogen which has a deleterious effect on the maintenance, viability and/or infectivity of said pathogen.

The nucleotide sequences introduced into a host or parent of a host are generally in the form of a chimeric genetic sequence.

In one embodiment of the present invention, the chimeric genetic sequence comprises multiple copies of a nucleotide sequence. In this context, the term “multiple copies” means two or more, such as but not limited to from about 2 to about 10, or from about 2 to about 5 or from about 2 to about 3.

In another embodiment, the chimeric genetic sequence comprises an inverted repeat separated by a “spacer sequence”. The spacer sequence may be a region comprising any sequence of nucleotides which facilitates secondary structure formation between each repeat, where this is required. The spacer sequence may comprise any combination of nucleotides or homologues, analogues or derivatives thereof which are capable of being linked covalently to a nucleic acid molecule. The spacer sequence may comprise a sequence of nucleotides of at least about 100-500 nucleotides in length, or alternatively at least about 50-100 nucleotides in length and in a further alternative at least about 10-50 nucleotides in length.

In a further embodiment, the chimeric genetic sequence comprises an inverted repeat wherein the spacer sequence comprises an intron sequence spliceable therefrom. In this context, the chimeric genetic sequence comprises intron/exon splice junction sequences, and an intron sequence may serve as a spacer sequence placed between the 3′ splice site of the first splice junction sequence and the 5′ splice site of another splice junction sequence.

The nucleotide sequences of the chimeric genetic sequence may be operably linked to one or more promoter sequences functional in a plant, vertebrate animal or fungus host. Alternatively, the nucleotide sequences are placed under the control of an endogenous promoter, normally resident in the host genome.

The chimeric genetic sequence of the present invention, under the control of an operably linked promoter sequence, may further be flanked by additional sequences which advantageously affect its transcription and/or the stability of a resulting transcript. Such sequences are generally located upstream of the operably linked promoter and/or downstream of the 3′ end of the dsDNA construct. Such a flanking sequence is referred to herein as an expression modulating sequence (EMS). Generally, an EMS occurs both upstream of the promoter and downstream of the 3′ end of the dsDNA construct, although an upstream EMS only is also contemplated.

Accordingly, another embodiment of the present invention is directed to a method for facilitating resistance or otherwise enhancing tolerance of a plant, vertebrate animal or fungal host to infection by a pathogen, said method comprising generating a host which comprises nucleotide sequences capable of directing synthesis of an RNA molecule, said nucleotide sequence selected from the list comprising:

(i) a nucleotide sequence transcribable to an RNA molecule comprising an RNA sequence which is substantially homologous to an RNA sequence encoded by a nucleotide sequence within the genome of said pathogen;

(ii) a reverse complement of the nucleotide sequence of (i);

(iii) a combination of the nucleotide sequences of (i) and (ii);

(iv) multiple copies of nucleotide sequences of (i), (ii) or (iii), optionally separated by a spacer sequence;

(v) a combination of the nucleotide sequences of (i) and (ii), wherein the nucleotide sequence of (ii) represents an inverted repeat of the nucleotide sequence of (i), separated by a spacer sequence;

(vi) a combination as described in (v), wherein the spacer sequence comprises an intron sequence sliceable from said combination; and

(vii) any of the above nucleotide sequences operably linked to a promoter and comprising one or more transcription enhancing sequences,

wherein upon ingestion by said pathogen of said RNA molecule or fragment or derivative thereof present in host cells, there is down-regulation of expression of said pathogen nucleotide sequence within the genome of said pathogen which has a deleterious effect on the maintenance, viability and/or infectivity of said pathogen.

Where the chimeric genetic sequence comprises an inverted repeat separated by a non-intron spacer sequence, upon transcription, the presence of the non-intron spacer sequence facilitates the formation of a stem-loop structure by virtue of the binding of the inverted repeat sequences to each other. The presence of the non-intron spacer sequence causes the transcribed RNA sequence (also referred to herein as a “transcript”) so formed to remain substantially in one piece, in a form that may be referred to herein as a “hairpin”. Alternatively, where the chimeric genetic sequence comprises an inverted repeat wherein the spacer sequence comprises an intron sequence, upon transcription, the presence of intron/exon splice junction sequences on either side of the intron sequence facilitates the removal of what would otherwise form into a loop structure. The resulting transcript comprises a double-stranded RNA (dsRNA) molecule, optionally with overhanging 3′ sequences at one or both ends. Such a dsRNA transcript is referred to herein as a “perfect hairpin”. The RNA molecules may comprise a single hairpin or multiple hairpins including “bulges” of single-stranded DNA occurring in regions of double-stranded DNA sequences.

Accordingly, in a preferred embodiment of the present invention, there is contemplated a method for facilitating resistance or otherwise enhancing tolerance of a plant, vertebrate animal or fungus host to infection by a pathogen, said method comprising generating a host comprising:

(i) a nucleotide sequence transcribable to an RNA molecule comprising an RNA sequence which is substantially homologous to an RNA sequence encoded by a nucleotide sequence within the genome of said pathogen;

(ii) a reverse complement of said nucleotide sequence; and

(iii) a spacer sequence separating said nucleotide sequence and reverse complement,

wherein the spacer sequence comprises an intron sequence; operably linked to a promoter and wherein upon ingestion by said pathogen of said RNA molecule or fragment or derivative thereof present in host cells, there is down-regulation of expression of said pathogen nucleotide sequence which has a deleterious effect on the maintenance viability and/or infectivity of said pathogen.

The present invention further contemplates genetic constructs for use in generating genetically-modified host organisms inclusive of plants, vertebrate animals or fungi resistant or exhibiting reduced susceptibility to pathogen infection, said genetic construct comprising a nucleotide sequence operably linked to a promoter wherein the nucleotide sequence is selected from:

(i) a nucleotide sequence transcribable to an RNA molecule comprising an RNA sequence which is substantially homologous to an RNA transcript from a gene in said pathogen;

(ii) the nucleotide sequence of (i) in the antisense orientation with respect to the promoter;

(iii) a combination of (i) and (ii) separated by a non-spliceable genetic element; and

(iv) a combination of (i) and (ii) separated by a spliceable genetic element.

The term “genetic element” in this context includes a spacer sequence.

Any of the sequences in (i) to (iv) may also comprise expression modulating sequences (EMS) which facilitate resistance of transgenetic sequences to methylation, thereby facilitating at least maintenance of expression levels. Suitable EMSs are disclosed in International Patent Application No. PCT/AU99/00434 (International Patent Publication No. WO 99/63068).

As indicated, the dsDNA construct of the present invention may comprise one or more nucleotide sequences operably linked to a promoter, functional in a plant, vertebrate animal or fungus or in a non-pathogenic microorganism.

In this regard, the present inventors have isolated Meloidogyne nucleotide sequences as set forth in SEQ ID NO:1; SEQ ID NO;2; SEQ ID NO:3; SEQ ID NO:4; SEQ ID NO:5; SEQ ID NO:6; SEQ ID NO:7 and SEQ ID NO:8, for use in genetic constructs of the invention.

In a particular embodiment, the invention contemplates use of SEQ ID NO: 1 and SEQ ID NO:3 in genetic constructs of the invention.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental, endogenous and/or environmental stimuli, or in a tissue-specific or cell-type-specific manner. A promoter is usually, but not necessarily, positioned upstream or 5′ of a structural gene, the expression of which it regulates. Furthermore, the regulatory elements comprising a promoter are usually positioned within 2 kb of the start site of transcription of the gene. The present invention extends to any promoter or promoter element recognized by one or more of a type I, II and/or III DNA polymerase. As is known in the art, some variation in this distance can be accommodated without loss of function. Similarly, the preferred positioning of a regulatory sequence element with respect to a heterologous gene to be placed under its control is defined by the positioning of the element in its natural setting, i.e. the genes from which it is derived.

In the present context, the term “promoter” is also used to describe a synthetic or fusion molecule, or derivative which confers, activates or enhances expression of a structural gene or other nucleic acid molecule, in a plant, animal or fungal cell or, where necessary, non-pathogenic microorganisms. Preferred promoters according to the invention may contain additional copies of one or more specific regulatory elements to further enhance expression in a cell, and/or to alter the timing of expression of a structural gene to which it is operably connected.

Promoter sequences contemplated by the present invention may be native to the host plant, animal or fungus or non-pathogenic microorganism to be transformed or may be derived from an alternative source, where the region is functional in the host plant, animal or fungus. Other sources for plants include the Agrobacterium T-DNA genes, such as the promoters for the biosynthesis of nopaline, octapine, mannopine or other opine promoters; promoters from plants, such as the ubiquitin promoter; tissue specific promoters (see e.g. U.S. Pat. No. 5,459,252 and International Patent Publication No. WO 91/13992); promoters from viruses (including host specific viruses) or partially or wholly synthetic promoters. Numerous promoters that are functional in mono- and dicotyledonous plants are well known in the art (see, for example, Greve, 1983; Salomon et al., 1984; Garfinkel et al., 1983; Barker et al., 1983); including various promoters isolated from plants (such as the Ubi promoter from the maize ubi-1 gene, see, e.g. U.S. Pat. No. 4,962,028) and viruses (such as the cauliflower mosaic virus promoter, CaMV 35S). Other sources for mammalian promoters include but are not limited to the promoters for the following genes: hypoxanthine phosphoribosyl transferase (HPTR), adenosine deaminase, pyruvate kinase and β-actin promoters. Exemplary viral promoters which function in eukaryotic cells include, for example, promoters from the simian virus (e.g. SV40), papilloma virus, adenovirus, human immunodeficiency virus (HIV), rous sarcoma virus, avian sarcoma virus, polyoma, cytomegalovirus, the long terminal repeats (LTR) of moloney leukemia virus and other retroviruses and the thymidine kinase promoter of herpes simplex virus. Other promoters are known to those of ordinary skill in the art. The promoters useful as gene expression sequences of the present invention also include inducible promoters. Inducible promoters are expressed in the presence of an inducing agent. For example, the metallothionein promoter is induced to promote transcription and translation in the presence of certain metal ions. Other suitable mammalian promoters include heterologous mammalian promoters, e.g. heat-shock promoters and the actin promoter.

Particular examples of promoters contemplated by the present invention include a tobacco 300 bp ToBRB7 promoter in the case of binary vector constructs and CaMV35S promoter.

Terms such as “operably connected”, “operably linked” or “in operable connection with” in the present context means placing a gene under the regulatory control of a promoter which then controls expression of the gene.

Furthermore, the chimeric genetic sequences of the present invention may also be operably linked to one or more of the following, functional in a plant, vertebrate animal or fungus or non-pathogenic microorganism: a 5′ non-coding region, a cis-regulatory region such as, for example, a functional binding site for transcriptional regulatory protein or translational regulatory protein, an upstream activator sequence, an enhancer element, a silencer element, a TATA box motif, a CCAAT box motif, or an upstream open reading frame, transcriptional start site, translational start site, and/or nucleotide sequence which encodes a leader sequence. In this regard, the EMSs contemplated herein may also be viewed as cis-acting regulatory sequences or an enhancer element.

The term “5′ non-coding region” is used herein in its broadest context to include all nucleotide sequences which are derived from the upstream region of an expressible gene, wherein the 5′ non-coding region confers or activates or otherwise facilitates, at least in part, expression of the gene.

The term “expression” is used in its broadest sense and includes transient, semi-permanent and stable expression, as well as inducible, tissue-specific, constitutive and/or developmentally-regulated expression. Expression encompasses generation of a transcript of a nucleotide sequence, with or without subsequent translation thereof.

In accordance with the present invention, upon the pathogen's ingestion of cells of a plant, vertebrate animal or fungus (including the serendipitous or intestinal ingestion of a non-pathogenic microorganism) expressing a suitable recombinant molecule, an RNA molecule is generated comprising an RNA sequence which is substantially homologous or complementary to an RNA molecule comprising an RNA sequence encoded by a gene in a cell or cells of a pathogen. A series of reactions is then set up which results in the effective degradation/removal of the substantially homologous RNA sequence encoded by a nucleotide sequence within the genome of the pathogen. The outcome is the silencing of a particularly targeted nucleotide sequence within the invading pathogen. In this context, “silencing” means the effective “down-regulation” of expression of the targeted nucleotide sequence and, hence, the elimination of the ability of the sequence to cause an effect within the pathogen's cell. This phenomenon is also variously known as “co-suppression” and/or “post-transcriptional gene silencing” (PTGS) and may be successfully effected via the introduction of synthetic recombinant molecules or transgenes, such as those contemplated by the present invention. In addition, the events of co-suppression or PTGS may also involve generation of RNAi, an inhibitory RNA molecule. The RNAi may be produced in the plant, vertebrate animal or fungal cell or it may be induced after the RNA molecule enters the pathogen's cells.

Without wishing to limit the mechanism of operation to one particular mode of action, it is proposed that the present “down-regulation” effect may be mediated through the action of a “dicer enzyme”, present in the cells of a plant, vertebrate animal or fungus. The expression of a dsDNA construct of the present invention results in the production of transcripts which, upon hybridization with an RNA sequence within the genome of said pathogen, yields dsRNA which is targeted by a dicer enzyme. The latter enzyme specifically restricts such molecules into small pieces of dsRNA of the order of about 19-25 nucleotides in length such as about 21 nucleotides in length. Such nucleotide-mers have particular predetermined overhanging 3′ ends at one or both ends of the molecule and are hence then targeted and degraded by an inherent cellular RNA-degrading mechanism, designed to remove unwanted foreign nucleic acid molecules from the cell. Hence, reference to “fragments or derivatives” of the RNA molecules includes fragments generated by, for example, dicer as well as RNAi or RNAi-like molecules comprising a fragment. Reference to RNAi and RNAi-like molecules is encompassed by the term “derivatives”.

As mentioned hereinbefore, it is contemplated that the method of the present invention may cause cells of a plant, vertebrate animal or fungus to express a dsDNA construct which results in transcripts that are substantially homologous to an RNA sequence encoded by a nucleotide sequence within the genome of an invading pathogen. Where the nucleotide sequence within the genome of an invading pathogen encodes a gene essential to the viability and/or infectivity of the pathogen, its down-regulation results in a reduced capability of the pathogen to survive and infect host cells. Hence, such down-regulation results in a “deleterious effect” on the maintenance viability and/or infectivity of said pathogen, in that it prevents or reduces the pathogen's ability to feed off and survive on nutrients derived from host cells. By virtue of this reduction in the pathogen's viability and/or infectivity, resistance and/or enhanced tolerance to infection by a pathogen is facilitated in the cells of a plant, vertebrate animal or fungus. Genes in the pathogen may be targeted at the mature (adult), immature (juvenile) or embryo stages.

In this context, “substantially homologous” to an RNA sequence encoded by a nucleotide sequence within the genome of an invading pathogen, means that the expressed transcript sequence will hybridize thereto under particular specified conditions. The term “hybridization” denotes the pairing of complementary nucleotide sequences to produce a DNA-DNA hybrid, a DNA-RNA hybrid, or an RNA-RNA hybrid. Complementary base sequences are those sequences that are related by the base-pairing rules. In DNA, A pairs with T and C pairs with G. In RNA U pairs with A and C pairs with G. Accordingly, the genetically modified host cells produce RNA molecules substantially homologous to pathogen-derived RNA transcripts.

Homology in this context includes sequence similarity or, more preferably, identity to 10 or more contiguous nucleotide sequences of an RNA sequence transcribed from a gene in the invading pathogen. Terms used to describe sequence relationships between two or more nucleotide sequences include “reference sequence”, “comparison window”, “sequence identity”, “percentage of sequence identity” and “substantial identity”. A “reference sequence” is at least 12 but frequently 15 to 18 and often at least 25 monomer units, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (1997). A detailed discussion of sequence analysis can be found in Unit 19.3 of Ausubel et al. (1998).

The terms “homology”, “sequence similarity” and “sequence identity” as used herein include, therefore, reference to the extent to which sequences are identical or functionally or structurally similar on a nucleotide-by-nucleotide basis over the comparison window. Similarity of a transcript sequence expressed from a dsDNA construct of the present invention to an RNA sequence encoded by a nucleotide sequence within the genome of an invading pathogen, may result in hybridization of one to the other, thereby resulting in the sequence of events referred to hereinabove, leading to the down-regulation of the nucleotide sequence within the genome of cells of an invading pathogen and its deleterious consequences.

Suitable nucleotide sequences within the genome of an invading pathogen include but are not limited to those which encode a gene essential to the viability and/or infectivity of the pathogen. Such genes may include genes involved in development and reproduction, e.g. transcription factors (see, e.g. Xue et al., 1993; Finney et al., 1988), cell cycle regulators such as wee-1 and ncc-1 proteins (see, e.g. Wilson et al., 1999; Boxem et al., 1999) and embryo-lethal mutants (see, e.g. Schnabel et al., 1991); proteins required for modelling such as collagen, ChR3 and LRP-1 (see, e.g. Yochem et al., 1999; Kostrouchova et al., 1998; Ray et al., 1989); genes encoding proteins involved in the motility/nervous system, e.g. acetycholinesterase (see, e.g. Piotee et al., 1999; Talesa et al., 1995; Arpagaus et al., 1998), ryanodine receptor such as unc-68 (see, e.g. Maryon et al., 1998; Maryon et al., 1996) and glutamate-gated chloride channels or the avermeetin receptor (see, e.g., Cully et al., 1994; Vassilatis et al., 1997; Dent et al., 1997); hydrolytic enzymes required for deriving nutrition from the host, e.g. serine proteinases such as HGSP-1 and HGSP-III (see, e.g. Lilley et al., 1997); parasitic genes encoding proteins required for invasion and establishment of the feeding site, e.g. cellulases (see, e.g. de Boer et al., 1999; Rosso et al., 1999) and genes encoding proteins that direct production of stylar or amphidial secretions such as sec-1 protein (see, e.g. Ray et al., 1994; Ding et al., 1998); genes encoding proteins required for sex or female determination, e.g. tra-1, tra-2 and egl-1, a suppressor of ced9 (see, e.g. Hodgkin, 1980; Hodgkin, 1977; Hodgkin, 1999; Gumienny et al., 1999; Zarkower et al., 1992); and genes encoding proteins required for maintenance of normal metabolic function and homeostasis, e.g. sterol metabolism, embryo lethal mutants (see, e.g. Schnabel et al., 1991) and trans-spliced leader sequences (see, e.g. Ferguson et al, 1996), pos-1, cytoplasmic Zn finger protein; pie-1, cytoplasmic Zn finger protein; mei-1, ATPase; dif-1, mitochondrial energy transfer protein; rba-2, chromatin assembly factor; skn-1, transcription factor; plk-1, kinase; gpb-1, G-protein B subunit; par-1, kinase; bir-1, inhibitor of apoptosis; mex-3, RNA-binding protein, unc-37, G-protein B subunit; hlh-2, transcription factor; par-2, dnc-1, dynactin; par-6, dhc-1, dynein heavy chain; and pal-1, homeobox. Such genes have been cloned from parasitic nematodes such as Meliodogyne and Heterodera species or can be identified by one of skill in the art using sequence information from cloned C. elegans orthologs (the genome of C. elegans has been sequenced and is available, see The C. elegans Sequencing Consortium (1998)). The only proviso in this regard is that, in this aspect of the present invention, the gene within the genome of an invading pathogen should not be substantially homologous to an endogenous gene of the host plant, vertebrate animal or fungus, including an endogenous gene of a human.

Examples of genes include pos-1, cytoplasmic Zn finger protein; pie-1, cytoplasmic Zn finger protein; mei-1, ATPase; dif-1, mitochondrial energy transfer protein; rba-2, chromatin assembly factor; skn-1, transcription factor; plk-1, kinase; gpb-1, G-protein B subunit; par-1, kinase; bir-1, inhibitor of apoptosis; mex-3, RNA-binding protein, unc-37, G-protein B subunit; hlh-2, transcription factor; par-2, dnc-1, dynactin; par-6, dhc-1, dynein heavy chain; pal-1, homeobox; and mex1, Zn finger protein.

Preferred genes or fragments thereof are mex1; skn-1; mei-1, dif-1; rba-1; gpb-1; and plk 1.

Nucleotide sequences of coding regions or part-coding regions of each of these genes are provided by SEQ ID NOS:1-8 respectively.

More preferred genes or fragments thereof are skn-1 (both M. incognita; SEQ ID NO:2; and M. javanica; SEQ ID NO:3) and M. incognita mex1 (SEQ ID NO:1).

Other known plant parasitic nematodes include but are not limited to seed or shoot gall nematodes (Anguina spp.) such as wheat gall nematode (Anguina tritici); ring nematodes (Criconema spp.) such as citrus spine nematode (Criconema civellae); bulb and stem nematodes (Ditylenchus spp.) such as potato rot nematode (Ditylenchus destructor) and rice stem nematode (Ditylenchus angustus); cyst nematodes (Globodera spp.) such as apple cyst nematode (Globodera mali); cyst or root nematodes (Heterodera spp.) such as cereal cyst and root nematode (Heterodera avenae), brassica root nematode (Heterodera cruciferae), wheat cyst nematode (Heterodera latipons) and sugar-cane cyst nematode (Heterodera sacchari); gall-forming or root-knot nematodes (Meloidogyne spp) such as, for example, barley root-knot nematode (Meloidogyne nassi), sorghum root-knot nematode (Meloidogyne acronea), southern root-knot nematode (Meloidogyne incognita) and Javanese root-knot nematode (Meloidogyne javanica); root-lesion or meadow nematodes (Pratylenchus spp.) such as carnation pin nematode (Pratylenchus dianthus), banana nematode (Pratylenchus musicola) and Thorne's root-lesion nematode (Pratylenchus thornei); stubby root nematodes (Trichodorus spp.) such as Christie's stubby root nematode (Trichodorus christiei); and stunt or stylet nematodes (Tylenchorhynchus spp.) such as, for example, sugar-cane stylet nematode (Tylenchorhynchus martini) and rice stunt nematode (Tylenchorhynchus martini) amongst others.

The term “facilitating resistance or otherwise enhancing tolerance”, as used herein, refers to any advantageous increased ability, conferred on a plant, vertebrate animal or fungus via the methods of the present invention, to resist succumbing to the disease-effects caused by infection by an organism which is usually pathogenic to the plant, vertebrate animal or fungus. Such effects may include, inter alia, stunted growth, wilting, reduced productivity and susceptibility to infection by other disease-causing agents. In some instances, even a relatively small increase in the ability to tolerate such infection may be beneficial and enable an improvement in, for example, yield of fruit from a plant. In other instances, the eventual development of full systemic resistance to infection may be desired. Examples of the latter include the ability of plants to resist and survive infection by usually lethal insect pests such as Helicoverpa; the ability of bovine animals to resist succumbing to the effects of, for example, the blood-sucking tick Boolphilus microplus; and the ability of ovine animals to resist succumbing to the effects of, for example, intestinal nematodes, inter alia.

A “pathogen” as used herein includes a nematode, insect, tick, arachnid or other creature which is capable of infecting or infesting host, and in particular, a plant, vertebrate animal or fungus.

Reference herein to a “nematode” refers to a member of the phylum Nematoda. Members of the family Heteroderidae are sedentary parasites that form elaborate permanent associations with the target host organism. They derive nutrients from cells of an infected organism through a specialised stylet. The cyst nematodes (genera Heterodera and Globodera) and root-knot nematodes (genus Meliodogyne), in particular, cause significant economic loss in plants, especially crop plants. Examples of cyst nematodes include, inter alia, H. avenae (cereal cyst nematodes), H. glycines (beet cyst nematode) and G. pallida (potato cyst nematode). Root-knot nematodes include, for example, M. javanica, M. incognita and M. arenaria. These pathogens establish “feeding sites” in the plant, by causing the morphological transformation of root cells into giant cells. Hence, nematode “infestation” or “infection” refers to invasion of and feeding upon the tissues of the host plant, vertebrate animal or fungus. Other nematodes that cause significant damage include the lesion nematodes such as Pratylenchus, particularly P. penetrans, which infects maize, rice and vegetables, P. brachyurus which infects pineapple and P. thornei which infects inter alia, wheat.

Because these pathogens establish “feeding sites” in plants via root cells, dsDNA constructs comprising cell-specific promoters and, more particularly, root-specific and root-tip-specific promoters, are especially preferred in this embodiment of the invention.

Insects that may cause damage and disease in plants belong to three categories, according to their method of feeding: chewing, sucking and boring. Major damage is caused by chewing insects that eat plant tissue, such as leaves, flowers, buds and twigs. Examples from this large insect category include beetles and their larvae (grubs), web-worms, bagworms and larvae of moths and sawflies (caterpillars). By comparison, sucking insects insert their mouth parts into the tissues of leaves, twigs, branches, flowers or fruit and suck out the plant's juices. Typical examples of sucking insects include but are not limited to aphids, mealy bugs, thrips and leaf-hoppers. Damage caused by these pests is often indicated by discolouration, drooping, wilting and general lack of vigour in the affected plant.

Insect pests causing plant disease include those from the families of, for example, Apidae, Curculionidae, Scarabaeidae, Tephritidae, Tortricidae, amongst others.

Damage and disease from insects also results in significant losses in the animal industries. Major insect pests of beef cattle, for example, include several species of biting flies, such as horn flies, stable flies and horse flies. The biting flies take blood directly from the cattle they attack and, in addition to causing worry, disease transmission and blood loss, also generate wounds that attract other pests.

In these and other aspects of the present invention, it is important that the presence of the nucleotide sequences transcribable from the dsDNA construct are neither harmful to cells of the plant, vertebrate animal or fungus in which they are expressed in accordance with the invention, nor harmful to an animal food chain and in particular humans. It is envisaged, in one embodiment, that the application of the invention will be in the generation of plants, animals and fungi cultivated by humans for the production of food. In this context, the ability to facilitate resistance or otherwise enhance tolerance of a plant, vertebrate animal or fungus to infection is advantageous in limiting losses of valuable food sources in world-wide agricultural and horticultural production. However, because the final use of the plant, vertebrate animal or fungus may be for human ingestion as food, the deleterious effects to be generated by the down-regulation of expression of the nucleotide sequence in an invading pathogen must occur only to the invading pathogen and not to the particular organism, whether plant, vertebrate animal or fungus.

In a preferred from, the method is applied to render a plant resistant or tolerant to infection by a pathogen. Most preferably, the plant is a crop plant. A “crop plant” means a plant species which is cultivated in order to produce a harvestable product. As used herein, the term “crop species” includes, but is not limited to, Abelmoschus esculentus (okra), Acacia spp., Agave fourcroydes (henequen), Agave sisalana (sisal), Albizia spp., Allium fistulosum (bunching onion), Allium sativum (garlic), Allium spp. (onions), Alpinia galanga (greater galanga), Amaranthus caudatus, Amaranthus spp., Anacardium spp. (cashew), Ananas comosus (pineapple), Anethum graveolens (dill), Annona cherimola (cherimoya), Apios americana (American potatobean), Arachis hypogaea (peanut), Arctium spp. (burdock), Artemisia spp. (wormwood), Aspalathus linearis (redbush tea), Athertonia diversifolia, Atriplex nummularia (old man saltbush), Averrhoa carambola (starfruit), Azadirachta indica (neem), Backhousia spp., Bambusa spp. (bamboo), Beta vulgaris (sugar beet), Boehmeria nivea (ramie), bok choy, Boronia megastigma (sweet boronia), Brassica carinata (Abyssinian mustard), Brassica juncea (Indian mustard), Brassica napus (rapeseed), Brassica oleracea (cabbage, broccoli), Brassica oleracea var Albogabra (gai lum), Brassica parachinensis (choi sum), Brassica pekensis (Wong bok or Chinese cabbage), Brassica spp., Burcella obovata, Cajanus cajan (pigeon pea), Camellia sinensis (tea), Cannabis sativa (non-drug hemp), Capsicum spp., Carica spp. (papaya), Carthamus tinctorius (safflower), Carum carvi (caraway), Cassinia spp., Castanospermum australe (blackbean), Casuarina cunninghamiana (beefwood), Ceratonia siliqua (carob), Chamaemelum nobile (chamomile), Chamelaucium spp. (Geraldton wax), Chenopodium quinoa (quinoa), Chrysanthemum (Tanacetum), cinerariifolium (pyrethrum), Cicer arietinum (chickpea), Cichorium intybus (chicory), Clematis spp., Clianthus formosus (Sturt's desert pea), Cocos nucifera (coconut), Coffea spp. (coffee), Colocasia esculenta (taro), Coriandrum sativum (coriander), Crambe abyssinica (crambe), Crocus sativus (saffron), Cucurbita foetidissima (buffalo gourd), Cucurbita spp. (gourd), Cyamopsis tetragonoloba (guar), Cymbopogon spp. (lemongrass), Cytisus proliferus (tagasaste), Daucus carota (carrot), Desmanthus spp., Dioscorea esculenta (Asiatic yam), Dioscorea spp. (yams), Diospyros spp. (persimmon), Doronicum sp., Echinacea spp., Eleocharis dulcis (water chestnut), Eleusine coracana (finger millet), Emanthus arundinaceus, Eragrostis tef (tef), Erianthus arundinaceus, Eriobotrya japonica (loquat), Eucalyptus spp., Eucalyptus spp. (gil mallee), Euclea spp., Eugenia malaccensis (jumba), Euphorbia spp., Euphoria longana (longan), Eutrema wasabi (wasabi), Fagopyrum esculentum (buckwheat), Festuca arundinacea (tall fescue), Ficus spp. (fig), Flacourtia inermis, Flindersia grayliana (Queensland maple), Foeniculum olearia, Foeniculum vulgare (fennel), Garcinia mangostana (mangosteen), Glycine latifolia, Glycine max (soybean), Glycine max (vegetable soybean), Glycyrrhiza glabra (licorice), Gossypium spp. (cottons), Grevillea spp., Grindelia spp., Guizotia abyssinica (niger), Harpagophyllum sp., Helianthus annuus (high oleic sunflowers), Helianthus annuus (monosun sunflowers), Helianthus tuberosus (Jerusalem artichoke), Hibiscus cannabinus (kenaf), Hordeum bulbosum, Hordeum spp. (waxy barley), Hordeum vulgare (barley), Hordeum vulgare subsp. spontaneum, Humulus lupulus (hops), Hydrastis canadensis (golden seal), Hymenachne spp., Hyssopus officinalis (hyssop), Indigofera spp., Inga edulis (ice cream bean), Inocarpus tugiter, Ipomoea batatas (sweet potato), Ipomoea sp. (kang kong), Lablab purpureus (white lablab), Lactuca spp. (lettuce), Lathyrus spp. (vetch), Lavandula spp. (lavender), Lens spp. (lentil), Lesquerella spp. (bladderpod), Leucaena spp., Lilium spp., Limnanthes spp. (meadowfoam), Linum usitatissimum (flax), Linum usitatissimum (linseed), Linum usitatissimum (Linola™), Litchi chinensis (lychee), Lotus corniculatus (birdsfoot trefoil), Lotus pedunculatus, Lotus sp., Luffa spp., Lunaria annua (honesty), Lupinus mutabilis (pearl lupin), Lupinus spp. (lupin), Macadamia spp., Mangifera indica (mango), Manihot esculenta (cassava), Medicago spp. (lucerne), Medicago spp., Melaleuca spp. (tea tree), Melaleuca uncinata (broombush), Mentha tasmannia, Mentha spicata (spearmint), Mentha X piperita (peppermint), Momordica charantia (bitter melon), Musa spp. (banana), Myrciaria cauliflora (jaboticaba), Myrothamnus flabellifolia, Nephelium lappaceum (rambutan), Nerine spp., Ocimum basilicum (basil), Oenanthe javanica (water dropwort), Oenothera biennis (evening primrose), Olea europaea (olive), Olearia sp., Origanum spp. (marjoram, oregano), Oryza spp. (rice), Oxalis tuberosa (oca), Ozothamnus spp. (rice flower), Pachyrrhizus ahipa (yam bean), Panax spp. (ginseng), Panicum miliaceum (common millet), Papaver spp. (poppy), Parthenium argentatum (guayule), Passiflora sp., Paulownia tomemtosa (princess tree), Pelargonium graveolens (rose geranium), Pelargonium sp., Pennisetum americanum (bulrush or pearl millet), Persoonia spp., Petroselinum crispum (parsley), Phacelia tanacetifolia (tansy), Phalaris canariensis (canary grass), Phalaris sp., Phaseolus coccineus (scarlet runner bean), Phaseolus lunatus (lima bean), Phaseolus spp., Phaseolus vulgaris (culinary bean), Phaseolus vulgaris (navy bean), Phaseolus vulgaris (red kidney bean), Pisum sativum (field pea), Plantago ovata (psyllium), Polygonum minus, Polygonum odoratum, Prunus mume (Japanese apricot), Psidium guajava (guava), Psophocarpus tetragonolobus (winged bean), Pyrus spp. (nashi), Raphanus satulus (long white radish or Daikon), Rhagodia spp. (saltbush), Ribes nigrum (black currant), Ricinus communis (castor bean), Rosmarinus officinalis (rosemary), Rungia klossii (rungia), Saccharum officinarum (sugar cane), Salvia officinalis (sage), Salvia sclarea (clary sage), Salvia sp., Sandersonia sp., Santalum acuminatum (sweet quandong), Santalum spp. (sandalwood), Sclerocarya caffra (marula), Scutellaria galericulata (scullcap), Secale cereale (rye), Sesamum indicum (sesame), Setaria italica (foxtail millet), Simmondsia spp. (jojoba), Solanum spp., Sorghum almum (sorghum), Stachys betonica (wood betony), Stenanthemum scortechenii, Strychnos cocculoides (monkey orange), Stylosanthes spp. (stylo), Syzygium spp., Tasmannia lanceolata (mountain pepper), Terminalia karnbachii, Theobroma cacao (cocoa), Thymus vulgaris (thyme), Toona australis (red cedar), Trifoliium spp. (clovers), Trifolium alexandrinum (berseem clover), Trifolium resupinatum (persian clover), Triticum spp., Triticum tauschii, Tylosema esculentum (morama bean), Valeriana sp. (valerian), Vernonia spp., Vetiver zizanioides (vetiver grass), Vicia benghalensis (purple vetch), Vicia faba (faba bean), Vicia narbonensis (narbon bean), Vicia sativa, Vicia spp., Vigna aconitifolia (mothbean), Vigna angularis (adzuki bean), Vigna mungo (black gram), Vigna radiata (mung bean), Vigna spp., Vigna unguiculata (cowpea), Vitis spp. (grapes), Voandzeia subterranea (bambarra groundnut), Triticosecale (triticale), Zea mays (bicolour sweetcorn), Zea mays (maize), Zea mays (sweet corn), Zea mays subsp. mexicana (teosinte), Zieria spp., Zingiber officinale (ginger), Zizania spp. (wild rice), Ziziphus jujuba (common jujube), soybean, corn, sunflower, rapeseed, wheat, barley, oat, rice and sorghum, tomato, potato, cucumber, onion, carrot, common bean, pepper and lettuce.

Particularly preferred crops include Nicotiana tabacum (tobacco) and horticultural crops such as, for example, Ananas comosus (pineapple), Lycopersicon esculentum (tomato) and Solanum tuberosum (potato). Preferably, the crop species exhibiting resistance or enhanced tolerance to pathogen infestation is a member of the species Ananas spp. or pineapple species.

Accordingly, a preferred embodiment of the present invention provides a method for facilitating resistance or otherwise enhancing tolerance of a plant to infection by a nematode, said method comprising generating a plant which comprises one or more nucleotide sequences transcribable to an RNA molecule which comprises an RNA sequence which is substantially homologous and/or complementary to an RNA sequence encoded by a nucleotide sequence within the genome of said nematode, such that upon ingestion by said nematode of said RNA molecule or a fragment or derivative thereof present in one or more cells of said plant, there is down-regulation of expression of said nematode nucleotide sequence which has a deleterious effect on the maintenance viability and/or infectivity of said nematode.

In an alternative preferred embodiment, there is provided a method for facilitating resistance or otherwise enhancing tolerance of a plant to infection or infestation by a nematode, said method comprising generating a substantially non-pathogenic microorganism which comprises one or more nucleotide sequences transcribable to an RNA molecule comprising an RNA sequence which is substantially homologous and/or complementary to an RNA molecule comprising an RNA sequence encoded by a nucleotide sequence within the genome of said nematode, such that upon ingestion by said nematode of said RNA molecule or a fragment or derivative thereof present in the microorganism associated with said plant, there is down-regulation of expression of said nematode nucleotide sequence which has a deleterious effect on the maintenance, viability and/or infectivity of said nematode.

The invention also contemplates another embodiment of a method for facilitating resistance or otherwise enhancing tolerance of a plant to infection or infestation by a nematode, said method comprising encapsulating in a synthetic matrix one or more RNA molecules comprising RNA sequences which is/are substantially homologous and/or complementary to an RNA molecule comprising an RNA sequence encoded by a nucleotide sequence within the genome of said nematode such that upon ingestion of said nematode when the synthetic matrix is applied to said plant, there is down-regulation of expression of said nematode nucleotide sequence which has a deleterious effect on the maintenance, viability and/or infectivity of said nematode.

In a particularly preferred embodiment, the present invention contemplates a method for facilitating resistance or otherwise enhancing tolerance of a pineapple plant to infection by a nematode or insect pest, said method comprising generating a pineapple plant which comprises one or more nucleotide sequences transcribable to an RNA molecule comprising an RNA sequence which is substantially homologous and/or complementary to an RNA sequence encoded by a nucleotide sequence within the genome of said nematode or insect pest, such that upon ingestion by said nematode or insect pest of said RNA molecule or a fragment or derivative thereof present in said pineapple plant cells, there is down-regulation of expression of said nematode or insect pest nucleotide sequence which has a deleterious effect on the maintenance, viability and/or infectivity of said nematode or insect pest.

Consistent with aspects and embodiments of the invention as hereinbefore described, the present invention may also be practised by applying to a host organism a biological or synthetic matrix comprising the RNA molecules which comprises RNA sequences homologous and/or complementary to RNA molecules in pathogen cells.

A “biological matrix” includes a microorganism such as a non-pathogenic or attenuated organism. The microorganism is engineered to synthesize the RNA molecule comprising RNA sequences homologous or complementary to pathogen sequences. Suitably, the microorganism is associated with the host in the sense that it is in sufficient physical proximity to the host to thereby be exposed to a pathogen infecting or infesting said host.

For example, microbial cells are engineered to produce the RNA molecules, which comprise RNA sequences which have substantial homology and/or complementarity to pathogen-derived RNA sequences, and applied to the surface of the pineapple plant. In this context, the preferred microbial cells are non-pathogenic or attenuated bacteria or yeast such as species of Pseudomonas, Agrobacterium, Bacillus and Rhizobium amongst many others. Insofar as the invention relates to protection of plants such as pineapples from nematode infection, the microorganism is suitably applied to the root system.

Still another embodiment provides the RNA molecules in a synthetic matrix which is applied to the host, such as to a surface of the plant (including a surface of the root system) or as an implant. A “synthetic matrix ” may be a polymer, adhesive, paint, fibrous material, binding agent, filler or coating which is applied to the host organism.. The synthetic matrix may have the RNA molecules encapsulated therein, bonded thereto or impregnated therewith in a manner that allows exposure of a pathogen to the RNA molecules.

In either case, the biological or synthetic matrix is applied to the surface of a plant, vertebrate animal or fungus. The term “surface” includes parts of a surface including root surfaces and under surfaces of leaves. Ingestion of plant, animal or fungal cells or tissue, or the contents thereof, by a pathogen results in the serendipitous ingestion of the biological or synthetic matrix comprising the RNA molecules.

Application may be via aerosol spray, chemical powder, vapour discharge, dip or aqueous medium.

Even still another embodiment involves modifying the plant such that a characteristic is introduced or altered in the plant which has the effect of reducing the ability of a nematode or other pathogen to maintain itself or grow on or in cells of the plant.

In accordance with a preferred embodiment of the present invention, a pineapple plant is genetically modified to express a dsRNA molecule. Methods for the production of transgenic plants are well known in the art. Such methods include inter alia micro-projectile bombardment (biolistics transformation), Agrobacterium-mediated transformation, electroporation, protoplast-mediated transformation and silicon carbide transformation.

Microparticles carrying a dsDNA construct of the present invention, which microparticles are suitable for the ballistic transformation of a cell, may be employed in transforming cells according to the present invention. The microparticle is propelled into a cell to produce a transformed cell. Where the transformed cell is a plant cell, a plant may be regenerated from the transformed cell according to techniques known in the art. Any suitable ballistic cell transformation methodology and apparatus can be used in practicing the present invention. Exemplary apparatus and procedures are disclosed in U.S. Pat. Nos. 5,122,466 and 4,945,050. When using ballistic transformation procedures, the dsDNA construct may be incorporated into a vector. Examples of microparticles suitable for use in such systems include 1 to 5 μm gold spheres. The dsDNA construct may be deposited on the microparticle by any suitable technique, such as by precipitation. Such ballistic transformation techniques are useful for introducing foreign genes into a variety of plant, vertebrate animal and fungal species, and are particularly useful for the transformation of monocotyledonous plants such as pineapple.

Vectors that may be used to carry out the present invention include Agrobacterium vectors. Numerous Agrobacterium vectors are known. See, e.g. U.S. Pat. Nos. 4,536,475, 4,693,977, 4,886,937, 5,501,967 and European Patent Application No. 0122791. In general, such vectors comprise agrobacteria, typically Agrobacterium tumefaciens, that carry at least one tumor-inducing (“Ti”) plasmid. When the agrobacteria are Agrobacterium rhizogenes, the plasmid is a root-inducing (or “Ri”) plasmid. The Ti (or Ri) plasmid contains DNA referred to as “T-DNA” that is transferred to the cells of a host plant when that plant is infected by the agrobacteria. In an Agrobacterium vector, the T-DNA is modified by genetic engineering techniques to contain the vector comprising the dsDNA construct, or the gene or genes of interest to be expressed in the transformed plant cells, along with any associated regulatory sequences. The agrobacteria may contain multiple plasmids, as in the case of a “binary vector system”. Such Agrobacterium vectors are useful for introducing foreign genes into a variety of plant species.

The combined use of Agrobacterium vectors and microprojectile bombardment is also known in the art (see, e.g. European Patent Nos. 0 486 233 and 0 486 234). Other vectors which may be used to transform plant tissue are well known in the art. The present invention incorporates the use of such vectors. The dsDNA constructs of the present invention may be comprised in Agrobacterium vectors, non-Agrobacterium vectors (particularly ballistic vectors), as well as other known vectors suitable for DNA-mediated transformation. Agrobacterium vectors are preferred.

Accordingly, one method for transforming cells of a monocotyledonous plant with genetic material comprises:

(a) obtaining an explant from said plant;

(b) co-cultivating the explant with Agrobacterium species having a T-DNA or T-DNA region comprising the genetic material to be transformed into the plant cells for a time and under conditions sufficient for the genetic material to transfer into the plant cells without said Agrobacterium overgrowing the plant cells; and

(c) selecting for the transformed plant cells and permitting the cells to form organogenic callus.

In a preferred embodiment, the present invention contemplates a method of genetically modifying a pineapple or related plant with one or more nucleotide sequences transcribable to an RNA molecule comprising an RNA sequence which is substantially homologous and/or complementary to an RNA sequence encoded by an nucleotide sequence within the genome of a pathogen such as a nematode or an insect pest, said method comprising:

(A) obtaining an explant from a pineapple or related plant to be genetically modified;

(B) co-cultivating the explant with Agrobacterium species having a T-DNA or T-DNA region comprising genetic material to be transferred into the pineapple or related cells for a time and under conditions sufficient for the genetic material to transfer to said cells;

(C) selecting for transformed pineapple or related cells and permitting the cells to form organogenic callus; and

(D) regenerating a pineapple or related plant from said organogenic callus.

Any plant tissue capable of subsequent clonal propagation, whether by organogenesis or embryogenesis, may be transformed with a vector of the present invention. The term “organogenesis”, as used herein, means a process by which shoots and roots are developed sequentially from meristematic centers; the term “embryogenesis”, as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g. apical meristems, axillary buds and root meristems) and induced meristem tissue (e.g. cotyledon meristem and hypocotyl meristem).

In a particular embodiment relating to pineapple, leaf explants are co-cultivated with Agrobacterium strain AGL0, comprising binary vectors engineered with the appropriate construct, as described in International Patent Publication No. WO 01/33943.

Plants of the present invention may take a variety of forms. The plants may be chimeras of transformed cells and non-transformed cells; the plants may be clonal transformants (e.g. all cells transformed to contain the chimeric genetic agent); the plants may comprise grafts of transformed and untransformed tissues (e.g. a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants (the term “subsequent generation” as used herein refers to T2 generation or thereafter) and the T2 plants further propagated through classical breeding techniques. Where the transgenic plant is bred with a plant that does not carry the same chimeric genetic agent to produce a hybrid plant, either plant may be the male of female parent. A dominant selectable marker (such as nptII) can be associated with the expression cassette to assist in breeding. Seeds may be collected from mature plants of the present invention in accordance with conventional techniques to provide seed that germinates into a plant as described herein.

As used herein, a transgenic plant also refers to those progeny of the initial transgenic plant which carry and are capable of expressing the chimeric dsDNA construct under the regulatory control of the qualitative and/or quantitative transcription control sequences herein described. Seeds containing transgenic embryos are encompassed within this definition. In the context of the present application, it is understood that the chimeric genetic agent is stably maintained in the genome of a transformed host plant cell, plant tissue and/or plant. Because seed formation occurs when flowers of a transgenic plant of the present invention are pollinated, the ordinarily skilled artisan can readily reproduce the plants of the invention.

Similar or analogous techniques are used to generate genetically modified vertebrate animals and fungi.

Reference to a vertebrate animal includes a livestock animal such as but not limited to a sheep, pig, cow, horse, donkey or goat, a laboratory test animal such as a mouse, rabbit, guinea pig or hamster or a companion animal such as a dog or cat. A vertebrate animal also includes avian species such as poultry birds, caged plants and game birds.

The present invention is, therefore, directed, in an another embodiment, to a vertebrate animal having resistance to nematode infection or infestation.

An animal produced by the method of the present invention is resistant to parasitic nematode infestation. Parasitic nematode infestations and infections contemplated herein include but are not limited to Ancylostoma infection (hookworm infection, Cutaneous Larva Migrans, CLM), including Ancylostoma caninum, Ancylostoma ceylanicum, Ancylostoma duodenale, Angiostrongylus infection (Angiostrongyliasis), Anisakis infection (Anisakiasis), Ascariasis (intestinal roundworns) including Ascaris lumbricoides, Ascaris suum, Baylisascaris infection (racoon roundworm), Brugia malayi, Capillaria infection (Capillariasis), Clonorchis infection (Clonorchiasis), Cryptosporidium infection (Cryptosporidiosis), Cysticercosis (Neurocysticercosis), Diphyllobothrium infection (Diphyllobothriasis), Dipylidium infection (dog or cat tapeworm infection), Echinococcosis (AHD, Alveolar Hydatid Disease), Fascioliasis (Fasciola infection), Fasciolopsiasis (Fasciolopsis infection), Giardia infection (Giardiasis), Gnathostoma infection (Gnathostomiasis), Heterophyes infection (Heterophyiasis), Leishmania infection (Leishmaniasis, Kala-azar), Litomosoides sigmodonti, Necator americanus, Onchocerca ochengi, Onchocerca volvulus, Opisthorchis infection (Opisthorchiasis), Ostertagia ostertagi, Parastrongyloides trichosuri, Paragonimus infection (Paragonimiasis), Pristionchus pacificus, Strongyloides ratti, Strongyloides stercoralis, Taenia infection (tapeworm infection), Teladorsagia trifurcata, Teladorsagia davtiani, Teladorsagia circumcincta, Toxocara infection (Toxocariasis, Ocular Larva Migrans, Visceral Larva Migrans), Toxocara canis Toxocariasis (Toxocara Trichinellosis (Trichinosis) Trichinella spiralis, Trichuris trichiura and Wuchereria bancroft.

The present invention is further directed to a genetically modified plant, vertebrate animal or fungus host wherein one or more cells of said genetically modified host comprise one or more nucleotide sequences transcribable to an RNA molecule comprising an RNA sequence which is substantially homologous and/or complementary to an RNA molecule comprising an RNA sequence encoded by a nucleotide sequence within the genome of said pathogen wherein said genetically modified host has increased resistance to infection by said pathogen.

Another embodiment of the present invention is directed to a genetically modified microorganism comprising one or more nucleotide sequences transcribable to an RNA molecule comprising an RNA sequence which is substantially homologous and/or complementary to an RNA molecule comprising an RNA sequence encoded by a nucleotide sequence within the genome of a pathogen wherein said genetically modified microorganism when applied to a plant, vertebrate animal or fungus host facilitates enhanced resistance of said host to infection by said pathogen.

Preferably, the genetically modified plant, vertebrate animal or fungus is resistant to or has enhanced tolerance against pathogens such as but not limited to nematodes and insects. In a particularly preferred embodiment, the genetically modified plant is a genetically modified pineapple plant.

Another aspect of the invention relates to novel, isolated nucleic acids which, for example, provide nucleotide sequences useful in genetic constructs for facilitating host resistance to pathogens.

The term “nucleic acid” as used herein designates single-stranded (ss) or double-stranded (ds) RNA inclusive of RNAi, mRNA, tRNA, cRNA and DNA inclusive of cDNA and genomic DNA and DNA-RNA hybrids.

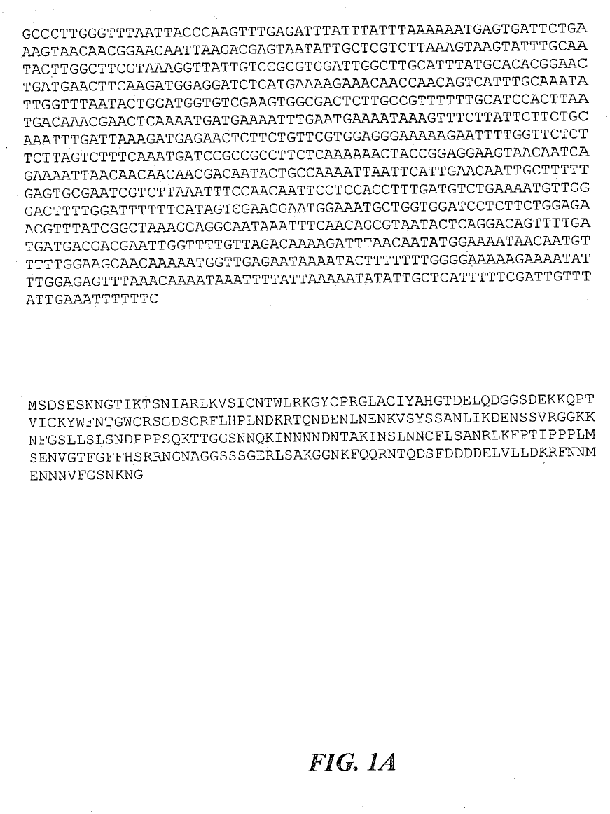

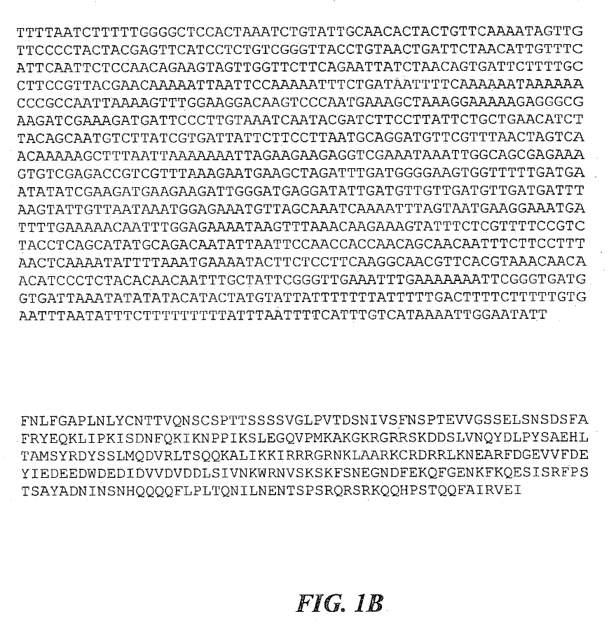

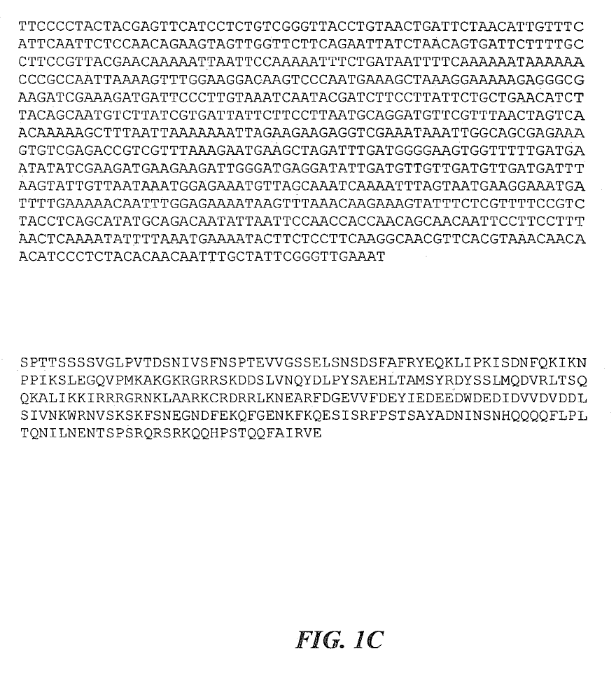

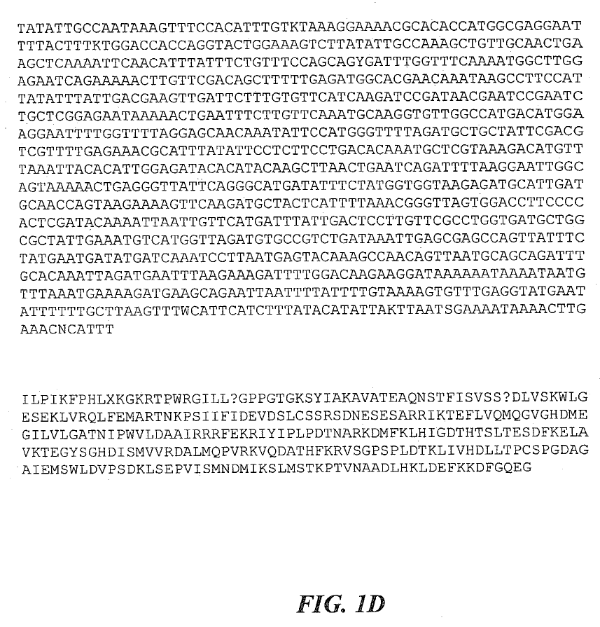

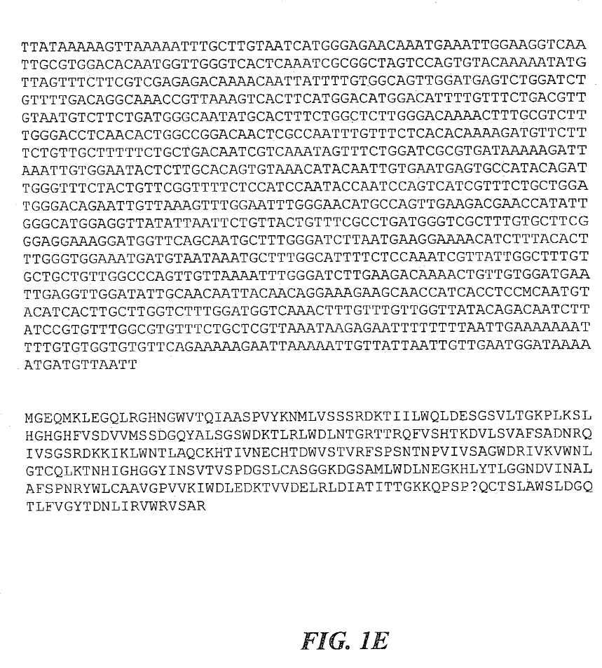

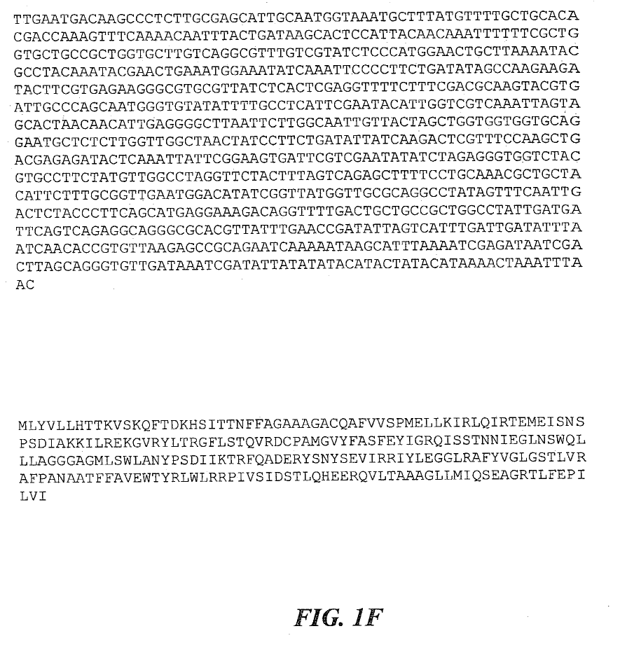

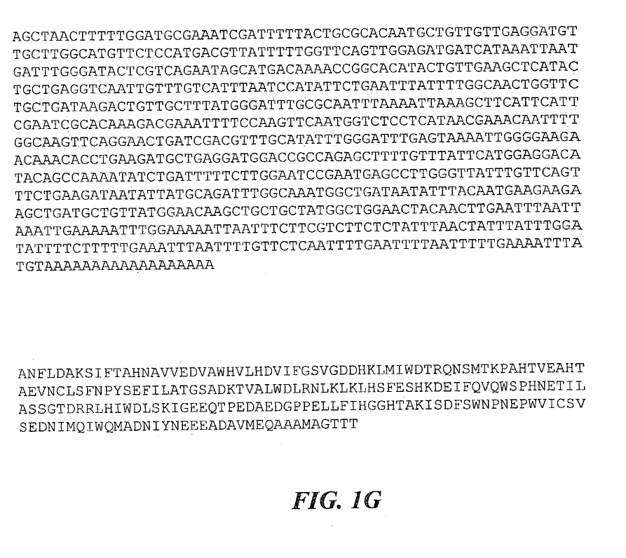

In particular embodiments, six Meloidogyne incognita genes (skn1, mei1, gpb1, plk1, dif1 and rba2) and two Meloidogyne javanica genes (skn1 and mex1) are provided in FIG. 1 and SEQ ID NOS: 1-8. In the case of M. javanica mex1 and M. incognita gbp 1, nucleotide sequence was obtained for the entire protein coding region of the respective genes. All deduced amino acid sequences (whether complete or partial) are set forth in FIG. 1 and SEQ ID NOS:9-16.

Also contemplated are fragments of the isolated nucleic acids and proteins of the invention.

Nucleic acid fragments may comprise at least 15 contiguous nucleotides and up to 100, 200, 500 or more contiguous nucleotides.

In a particular embodiment, said nucleic acid fragment is suitable for use in a genetic construct of the invention.

In other particular embodiments, said nucleic acid fragment is suitable for use as a primer or probe as is well known in the art.

Typically, said primer has 15-70 contiguous nucleotides of any one of SEQ ID NOS:1-8.

This aspect of the invention also contemplates homologous nucleic acids and encoded proteins inclusive of orthologous nucleic acids and proteins isolated from other organisms, whether nematodes or otherwise.

In one embodiment, homologues and/or orthologues of the invention have at least 50%, preferably at least 70% or more preferably a least 80 or 90% sequence identity (as hereinbefore defined) with any one of SEQ ID NOS:1-8. According to this embodiment, sequence identity is preferably compared over at least 25, more preferably at least 100 and even more preferably at least 200 contiguous nucleotides of any one of SEQ ID NOS:1-8.

In another embodiment, said homologue is substantially complementary to any one of SEQ ID NOS:1-8.

In yet another embodiment, homologues and/or orthologues of the invention hybridize with any one of SEQ ID NOS:1-8, at least under low stringency conditions, preferably under at least medium stringency conditions and more preferably under high stringency conditions.

The concepts of “hybridization”, “stringency” and “stringent conditions” are well known in the art, such as described in Chapters 2.9 and 2.10 of CURRENT PROTOCOLS IN MOLECULAR BIOLOGY Eds. Ausubel et al., (1995-2001)and in particular at pages 2.9.1 through 2.9.20.

By way of example, reference herein to low stringency conditions includes and encompasses:

(i) from at least about 1% v/v to at least about 15% v/v formamide and from at least about 1 M to at least about 2 M salt for hybridisation at 42° C., and at least about 1 M to at least about 2 M salt for washing at 42° C.; and

(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Medium stringency conditions include and encompass:

(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and

(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

High stringency conditions include and encompass:

(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 68° C. for about 20 minutes.

The present invention is further described by the following non-limiting Examples.

### EXAMPLE 1

### Inactivation of Critical Nematode Genes by RNA Interference Methods

C. elegans Bioassay for Rapid Screening of Potential Target Genes

A quick and efficient bioassay is critical to evaluate the effect on viability of down-regulating selected genes in nematodes. It is not practical, however, to develop a bioassay for M. javanica since it is a facultative parasite. To overcome this problem, the free living nematode C. elegans is used. Timmons & Fire (1998) have shown that C. elegans genes can be inactivated by feeding E. coli bacteria actively expressing dsRNA.

C. elegans genes were cloned into a high copy number plasmid at a site between two opposing T7 polymerase promoters. Expression from the T7 polymerase results in the production of dsRNA in the bacterium.

For the C. elegans feeding bioassay, cloning and bacterial transformation steps were performed using a standard laboratory strain of E. coli (DH10B from Life Technologies Inc.). Constructs were then transferred to mutant bacterial strain E. coli HT115 (DE3) that is deficient in RNaseIII. This allows inducible expression of large amounts of dsRNA (Timmons et al., 2001).

The experimental procedure of Kamath et al., (2000) was used for the C. elegans bioassay.

Identification of Potential Gene Targets in C. elegans

Genes which show high conservation among nematodes but not with animal or plant genes are selected and cloned from C. elegans. Potential genes are evaluated based on size, homology with plant or human genes and availability. Table 1 shows potential ESTs for C. elegans genes. The effect of the selected genes was validated using the C. elegans bioassay (by bacterial feeding). Two target genes were selected that are essential for normal embryo development.

Eight target genes (mex-1, gpb1, mei1, pos1, pie1, rba-2, skn1 and unc37) produced a 100% embryo lethal phenotype in the C. elegans bio-assay. These genes were initially ranked into groups 1 (mei1, pos1, pie1), group 2 (rba-2, skn1), group 3 (gpb1) and group 5 (unc37). This ranking system was based on size and homology with plant or human genes. The pie1 and mex-1 genes have not previously been reported to give an embryo lethal phenotype in RNAi experiments.

Cloning of Orthologous Genes in M. javanica and M. incognita

The present inventors have used a bioinformatics approach to clone Meloidogyne homologues of the target C. elegans genes rather than the traditional library screening approach.

The Washington University Genome Sequence Centre has a Parasitic Nematode EST Project from which 18,898 Meloidogyne sequenced clones have been described on its public database.

The present inventors have obtained 25 ESTs covering 8 target genes (Table 2).

Six Meloidogyne incognita genes (skn1, mei1, gpb1, plk1, dif1 and rba2) and two Meloidogyne javanica genes (skn1 and mex1) were fully sequenced and the sequences are provided in FIG. 1 and SEQ ID NOS: 1-8). In the case of M. javanica mex1 and M. incognita gbp 1, nucleotide sequence was obtained for the entire protein coding region of the respective genes. All deduced amino acid sequences (whether complete or partial) are set forth in FIG. 1 and SEQ ID NOS:9-16.

When the pos1/pie1 like EST was fully sequenced, the predicted protein sequence had higher similarity with MEX1 than with POS1/PIE1. All three genes encode structurally similar transcription factors that are expressed during the first three cell divisions of C. elegans embryogenesis. Therefore the M. javanica EST clone rk10c12.y1 is probably a mex1 homologue.

The sequenced Meloidogyne genes were grouped into low (mex1, skn1, dif1), medium (mei1, gpb1, plk1) or high (rba2) according to nucleotide sequence homology with other genes on the public databases.

The M. javanica and C. elegans gene sequences were compared, but there were no regions of high homology. The Meloidogyne genes mex1 and skn1 had low sequence homology with other genes and their C. elegans homologue produced a 100% embryo lethal phenotype in the bio-assay. These two genes were selected as the nematode targets.

The two genes mei1 and gpb1 genes are potentially suitable although less preferred targets for similar reasons.

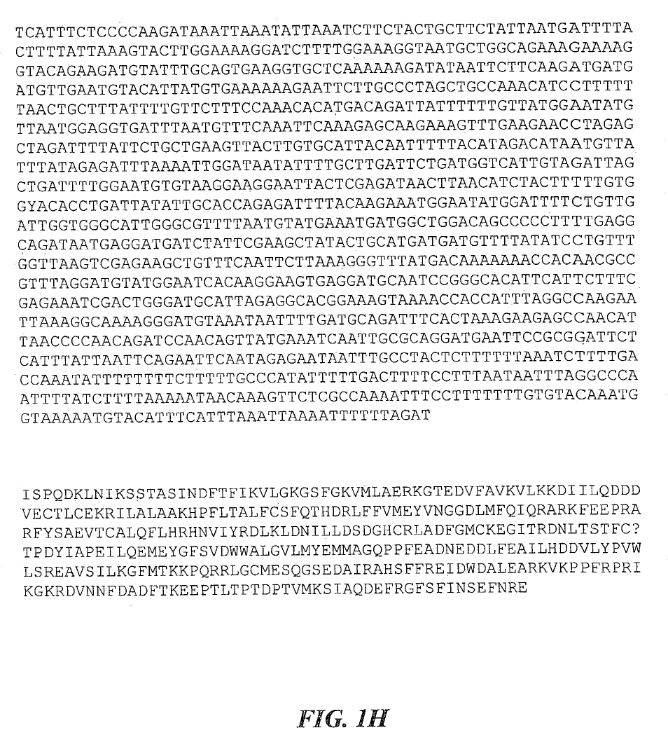

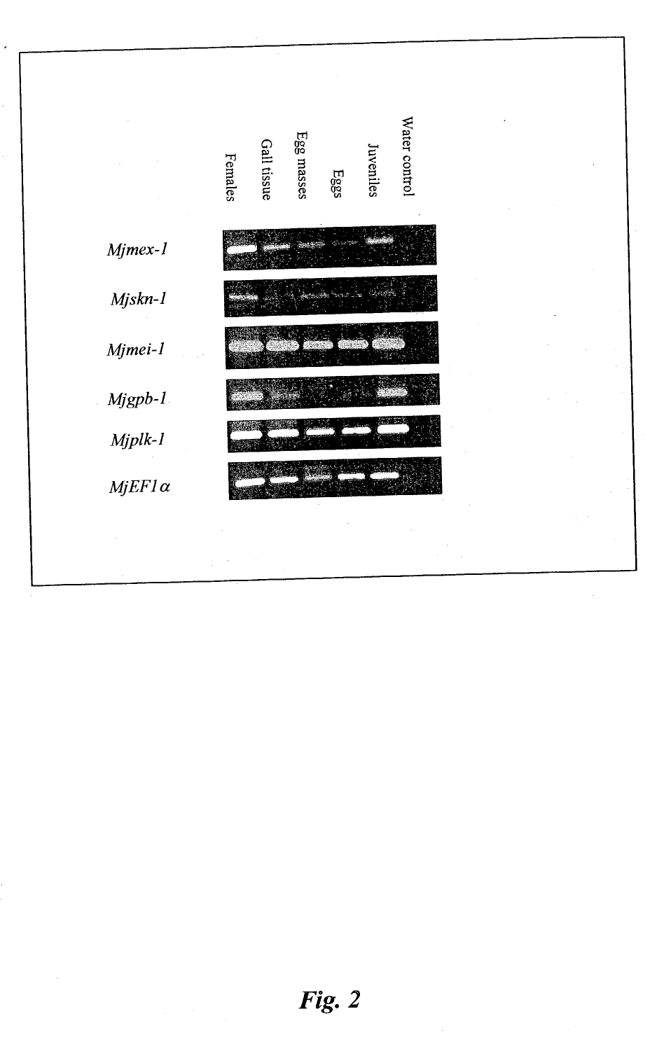

RT-PCR analysis of candidate gene expression in M. javanica (FIG. 2) indicated that RNAi inactivation of these genes may affect juvenile and adult stages as well as the developing embryos. The MjEF1 gene is an elongation factor that is expressed constitutively and used as a control in the experiment.

Assav M. javanica Genes in C. elegans Bioassay

RNAi is sequence specific and requires high nucleotide sequence identity (approx >87%) between the trigger and target sequences. Consequently, for a Meloidogyne gene to work in a C. elegans bio-assay the genes must share high sequence identity over at least 25 consecutive bases. The Meloidogyne genes that were sequenced did not have the required minimum level of identity. For example, M. javanica MEX1 protein was only about 18, 11 and 13% identical to C. elegans MEX1, POS1 and PIE1 proteins respectively, with even less identity at the nucleotide sequence level. Those Meloidogyne genes that have this property also have regions of high nucleotide homology with human genes and other organisms. This would exclude them as candidates for binary vectors. Therefore, the Meloidogyne genes exemplified herein were not tested in the C. elegans bio-assay.

Preparation of Constructs for Tobacco Transformation

Two nematode target genes: M. javanica mex1 and M. javanica skn1, were cloned from M. javanica egg first strand cDNA by PCR using the information from the fully sequenced Washington clones.

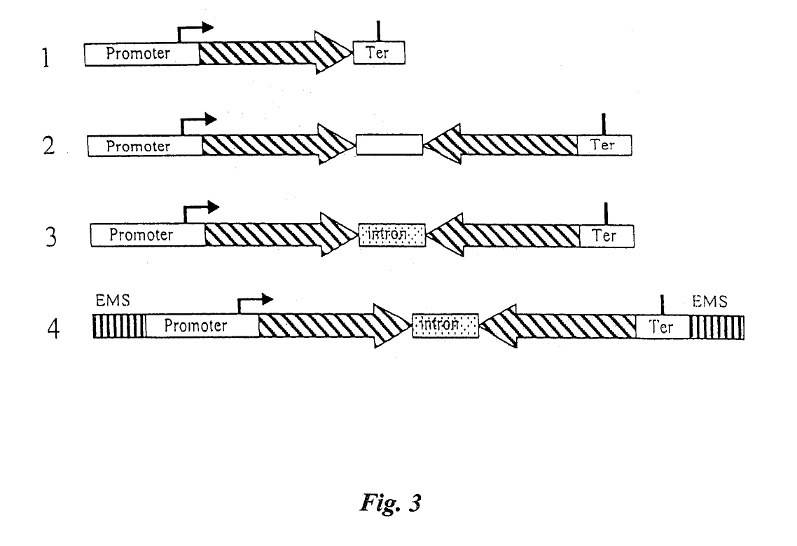

Four different types of construct were prepared (FIG. 3):

(1) Conventional sense. This construct serves largely as a control, although it is possible some tobacco lines transformed with this construct might express small amounts of dsRNA or co-suppressing RNA;

(2) Hairpin. This construct is specifically designed to express a self-complementary RNA, which potentially forms dsRNA (Chuang and Meyerowitz, 2000).

(3) Perfect hairpin. It has been recently demonstrated that expressing RNA as a “perfect hairpin” in transgenic plants results in extremely high frequencies of co-suppression in plants (Smith et al., 2000). By testing this class of construct, the inventors determine whether a co-suppressing RNA can inactivate a parasitic nematode RNA; and

(4) Perfect hairpin buffered by EMS sequences. A construct “buffered” by EMSs (Expression Modulating Sequences). These sequences when placed near a gene can inhibit co-suppression. Potentially, dsRNAs expressed in constructs flanked by these EMSs may not co-suppress efficiently and remain as dsRNA.

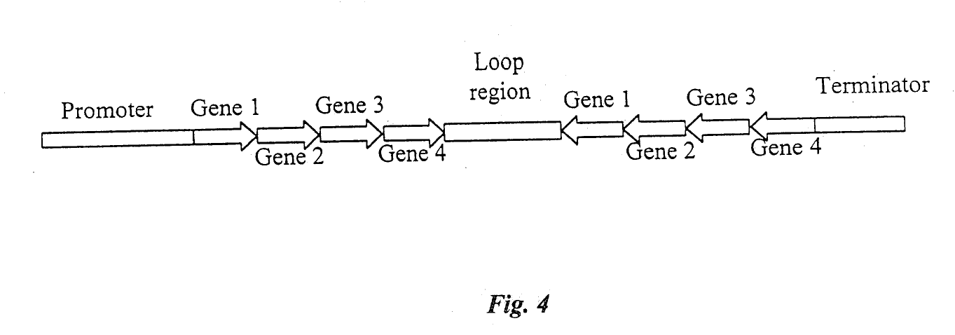

A further construct type comprising multiple pathogen genes or gene fragments is shown in FIG. 4.

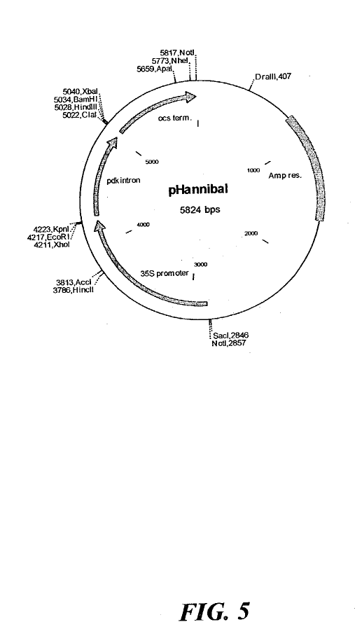

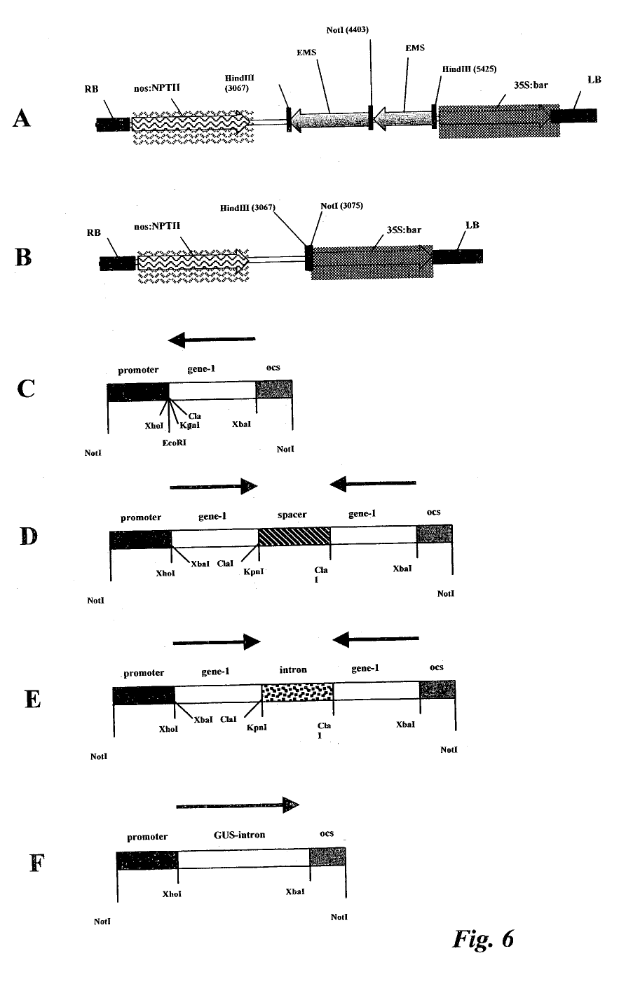

Vectors for plant transformation were prepared in two stages. Intermediates were sequenced and subjected to restriction enzyme analysis to confirm their integrity. The initial cloning steps involving target genes, spacer, intron and promoters were carried out in the plasmid pHannibal (Wesley et al., 2001; FIG. 5). Once the components were fully assembled the NotI cassette was then subcloned into one of two binary vectors. All pHannibal NotI cassettes (FIG. 6C-F) were cloned into the binary vector pUQC477 (FIG. 6B). In addition, the perfect hairpin constructs (FIG. 6E) were also cloned into the pUQC136 (FIG. 5A) binary vector containing EMS sequences (UQ14).

The tobacco 300 bp TobRB7 promoter was chosen for the binary vectors because it has been shown to be inducible by root-knot nematode infection and is expressed only at nematode feeding sites (Opperman et al., 1994). We also used the CaMV35S promoter which drives transgene expression throughout the plant.

The LEMMI9 promoter can also be used to direct target gene expression at nematode feeding sites (Escobar et al., 1999).

A 3′ coding region of the uidA gene (GUS; β-glucuronidase) was used as a spacer of the same length as the pdk intron (FIG. 6D).

GUS marker gene constructs (FIG. 6F) were prepared with each of the promoters. These constructs were used as controls to monitor promoter expression during experiments.

The target genes mex1 and skn1 were each assembled into the vectors in FIG. 6C-E. This was done with both the TobRB7 and CaMV35S promoters.

Production of Transgenic Tobacco Plants

Tobacco plants were transformed with the different constructs with the aim of producing large amounts of dsRNA such that, upon feeding, the targeted M. javanica genes are inactivated.

All constructs were prepared in E. coli DH10B and then transferred to Agrobacterium tumefaciens strain LBA4404 by triparental mating using the E. coli helper strain pRK2013.

A rapid flowering line of tobacco, Nicotiana tabacum Ti68; (McDaniel et al., 1996) was transformed with A. tumefaciens containing the constructs using the leaf disc method. The present inventors have found that this line is susceptible to M. javanica and produces large numbers of eggs.

An outline of the method used for tissue culture and selection of Agrobacterium-transformed tobacco tissue is as follows.

(1) Agrobacterium suspension (5 ml per 20 explants).

(2) Transform 40-80 tobacco explants per construct (20 explants per MSO plate), 4 days in dark.

(3) Wash off Agrobacterium with cefotaxime, blot explants dry and put onto M9 medium (20 explants per plate).

(4) Subculture explants every 2 weeks, keeping flat, as explant develops shoots and expands reduce density on plate (eg. 4 to 6 per plate).

(5) When good well-formed shoots, transfer single shoots without callus to rooting medium in plates. Number all shoots from same explant with same number. This is because it is assumed that the shoots from the same explant are not independent transgenic events. Subculture the remainder of explant on KMSO medium and regularly check for shoots.

(6) Subculture every 2 weeks on fresh medium.

(7) When roots appear on individual shoots, mark the group of shoots on the plate and transfer rooted shoot to rooting medium in Universal container.

(8) When this shoot elongates, take nodal cuttings into rooting medium in bunzel containers.

(9) Record those shoots rooting and sample one of the propagated clones for PCR analysis.

(10) Record PCR results and mark positive propagated shoots.

(11) Rooted, PCR positive propagated shoots ready for deflasking.

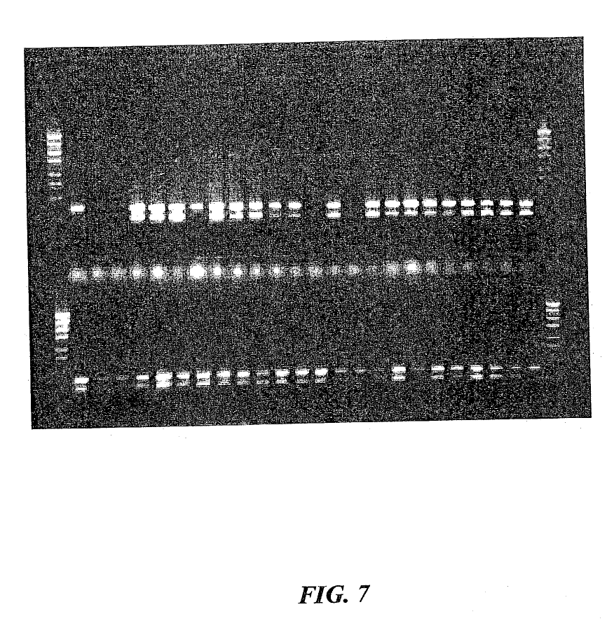

Many transgenic lines were produced. These were tested by PCR to check that they were transgenic (i.e. contained the NPTII and Bar selectable marker genes) and contained either the mex1 or skn1 target genes. An example is given in FIG. 7. The DNA size markers are in the side lanes. The PCR products from a duplex reaction amplifying a tubulin control (top band) and the NPTII transgene (bottom band) have been separated by gel electrophoresis. Wild-type controls are in the first lane (top panel on left) and last lane (bottom panel on right). Most of the putative transgenic tobacco are positive

Fifteen confirmed transgenic and independent lines per construct and three replicates per line were challenged with M. javanica nematodes in a glasshouse trial.

Glasshouse Trial of Transgenic Tobacco

Plants were deflasked and processed in batches to allow the harvest of all plants in a batch at the same time. The size of these batches was about 53. Each batch included 6 wild-type control plants, 3 of which were infected and the other 3 were non-infected controls. Precautions were taken to keep the non-infected tobacco nematode free during the trial. There were 3 replicates per transgenic line, which were kept in different batches, and 15 lines per construct.

The standard procedure for each batch included the following:

Deflask tobacco plants from tissue culture containers to 10 cm pots with sandy compost

Plants in batches of 53 including 3 wild-type controls that are not to be infected

Arrange pots randomly in each batch

Allow plants to acclimatize, initial at high humidity, and develop a healthy root system (2 weeks)

Infect each plant with 10,000 M. javanica eggs prepared under standard conditions (Assuming 20% egg hatch and a 10 cm pot volume of 550 cm⁻³ gives 3.6 juveniles per cm⁻³ of soil)

Leave for a set time of 6 weeks and then harvest (This will allow 1 generation to be completed and the second generation to produce eggs at 25-30° C.)

Aerial tissue is then harvested from each plant as follows:

Weighed

Save seed pods in separate labelled paper bag

Save young leaves in a tube and freeze in liquid nitrogen, store on dry ice and then keep at −80° C. for molecular analysis

Root tissue is harvested from each plant as follows:

Weigh

Score gall index

Strip eggs from roots

Save some gall tissue in a tube and freeze in liquid nitrogen, store on dry ice and then keep at −80° C. for future molecular analysis

Eggs are harvested from each plant as follows:

Estimate egg hatch percentage of inoculum used

Estimate number of eggs produced per plant and reproduction factor

Hatch known number of eggs and score juveniles and estimate egg viability

Save some eggs in a tube and freeze in liquid nitrogen, store on dry ice and then keep at −80 C. for future molecular analysis

Samples kept for molecular analysis are used for the following assays:

Leaf tissue used for Southern blot analysis to check T-DNA integration and transgene copy number

RT-PCR of first strand cDNA prepared from eggs and gall tissue containing adult female nematodes using primers to the target genes designed to sequences outside of the region used for making the RNAi constructs. This will allow amplification of the endogenous nematode gene and not the target gene introduced into the plant. Results quantified by normalization of the data to a constitutively expressed gene such as the M. javanica elongation factor 1.

RT-PCR of first strand cDNA is prepared from plant tissues (gall, non-gall root, leaf) using primers to the transgenes designed to sequences only in the RNAi constructs. This will allow amplification of the target genes introduced into the plant and not the endogenous nematode genes. Results are quantified by normalization of the data to a constitutively expressed gene such as the tobacco elongation factor 1.

Detection and specificity of small interfering RNAs (siRNAs) in plant and nematode tissues. The siRNAs are associated with the RNAi/PTGS response and trigger the sequence specific degradation of mRNAs.

Northern blot analysis of target gene expression in various plant and nematode tissues.

Assessing methylation status of T-DNA regions in genomic DNA from plant gall and leaf tissue.

The level of nematode resistance in the transgenic plants is judged by comparison of various performance factors to those of infected and non-infected wild-type control plants. These performance factors include plant biomass, number of eggs produced per plant, nematode reproduction factor, nematode gall index and egg viability.

### EXAMPLE 2

### Application of RNAi Methods to Control other Plant Pests

RNAi methods have now been proven to work in a variety of organisms aside from nematodes, including insects such as Drosophila (Kennerdell and Carthew, 1998). RNAi methods are applied, therefore, towards controlling other plant pests such as insects.

Model System

The inventors use lettuce and the Helicoverpa armigera caterpillar as the plant and insect model system. Lettuce has a simple, efficient and fast transformation system. A transient expression system is developed in lettuce by Agrobacterium infiltration which allows optimization of a bioassay method. Lettuce leaves infiltrated with Agrobacterium tumefaciens can quickly and efficiently express the genes contained between the border sequences.

Identification and Cloning of Important Genes in Helicoverpa armigera

Genomic and cDNA libraries are constructed from H. armigera. Genes targeted include those important for nutrition and active in the intestinal gut, such as proteases and transporters. It is assumed that the most likely place for RNAi to operate is in the intestinal tract since the method of delivery of the dsRNA will be via ingestion of the plant tissue by the worm. Other possible genes are those important for the survival of the animal, such as those implicated in cell division (cdc family), amino acid synthesis, cellular respiration and the like. Genes are cloned by RT-PCR for those proteins with conserved regions or identified by heterologous screening of the libraries using probes from related organisms.

Development and Optimization of a Bioassay to Ascertain the Effectiveness of Targeted Genes

Two different bioassays are used:

(A) Agrobacterium leaf infiltration. Only constructs containing a perfect hairpin are used in this bioassay. Genes are under the control of a strong constitutive promoter resulting in the production of large amounts of dsRNA. The kinetics of dsRNA accumulation are determined in the plant tissue. Challenge experiments are performed by placing caterpillars in plant leaves for feeding. Feeding is performed on plant tissues at the time in which dsRNA content is peaking (as determined by the kinetics experiments). Leaves are changed at regular intervals to ensure that the amount of dsRNA in the plant tissues is as high as possible. Caterpillars are continually monitored and health parameters evaluated (including weight evolution).

(B) Bacterial production of dsRNA. The effectiveness of bacterial preparations for delivery of dsRNA is tested. Bacterial strains comprising constructs that result in the production of dsRNA are prepared as described by Tabara et al. (1998). Lettuce leaves are vacuum infiltrated with a bacterial preparation and caterpillars allowed to feed on the leaves. RNA extractions are prepared on infiltrated leaves to quantify the levels of dsRNA in the total RNA population (consisting of plant and bacterial RNA). Caterpillars are monitored as described above.

Preparation of Binary Constructs for Transformation

The same constructs described above for the nematode approach are used, with one major difference. Due to the nature of the pest being targeted, the gene only needs to be active in the wounded tissue being eaten by the caterpillar. For this purpose, a strong wound-inducible promoter with rapid kinetics is used. The use of such a promoter ensures that the dsRNA is only produced on those tissues being directly attacked. Fast, transient and localized expression is also used to address the possibility of internal down-regulation of the dsRNA which can occur under the control of a strong constitutive promoter.

TABLE 1
C. elegans EST details

| | Gene | Gene | EST | EST | GenBank | GenBank |
|---|---|---|---|---|---|---|
| Number | Name | Function | Requested | Size | (5′) | (3′) |
| 1 | pos-1 | cytoplasmic Zn finger protein | YK667H9 | Full | AV197562 | AV184679 |
| | | | YK173F3 | Missing ATG | C09622 | C08035 |
| 2 | hlh-2 | Transcription factor E2-A like | YK492C11 | Full | C51185 | C38378 |
| | | | YK337C11 | Missing ATG | C43951 | C32936 |
| 3 | mei-1 | ATPase | YK746G9 | Full | AU112773 | AU116617 |
| | | | YK514H5 | Missing ATG | AV187703 | AV176568 |
| 4 | rba-2 | Chromatin assembly factor | YK744G6 | Full | AU112606 | AU116449 |
| | | | YK631C3 | Missing ATG | AV194580 | AV182038 |
| 5 | gpb-1 | G-protein B subunit | YK607A6 | Full long 5′UTR | AV192576 | |
| 6 | par-1 | Kinase Ser/Thr | YK399D8 | 5′ only | C45569 | |
| | | | YK27H10 | 3′ only | D35989 | |
| 7 | par-2 | ATP/GTP binding sites and Zn finger | YK628A12 | Full | AV194323 | |
| 8 | par-6 | | YK266F12 | Full | C41329 | |
| 9 | skn-1 | transcription factor | YK388D7 | 3′ only | AV201463 | |
| 10 | dnc-1 | Dynactin | YK88H6 | 3′ only | D67955 | |
| 11 | bir-1 | Inhibitor of apoptosis | CESAB78F | 3′ only | T02295 | |
| 12 | pal-1 | Homeobox | YK723H6 | Full | AU111102 | |
| | | | YK721A3 | Missing ATG | AU110664 | |
| 13 | dif-1 | Mitochondrial energy transfer proteins | YK640E4 | Full | AV195324 | AV182628 |
| | | | YK603G8 | Missing ATG | AV192305 | AV180189 |
| 14 | plk-1 | Polo-like kinase | YK622C7 | Full | AV193850 | AV181585 |
| | | | YK320C8 | Missing ATG | C63391 | C53517 |
| 15 | dhc-1 | Dynein heavy chain | YK27B2 | 3′ only | | |
| 16 | mex-3 | RNA-binding protein | YK504H4 | Full | AV186857 | |
| | | | YK269E8 | Missing ATG | C41704 | |
| 17 | unc-37 | G-protein B subunit | YK707F10 | Full | AU109505 | |
| | | | YK434B9 | Missing ATG | C47239 | |
| 18 | pie-1 | Cytoplasmic Zn finger protein | YK712C8 | Full | AU109889 | |
| | | | YK637D9 | Missing ATG | AV195086 | |
| 19 | dnc-2 | as dnc-1 | YK393F9 | Full | C45416 | C34241 |
| 20 | bir-2 | as bir-1 | YK319E9 | 3′ only | C42784 | |
| 21 | DRG-like | | YK364H8 | 5′ only | C69264 | |
| 22 | DRG-like | | YK211A3 | Full | C39472 | |
| 23 | mex-1 | zinc finger protein | | | | |

TABLE 2
Meloidogyne ESTs on database

| | | | | Tblastn with | | | |
|---|---|---|---|---|---|---|---|
| | | | | C. elegans | | | |
| Number | Gene | EST | Number | Score and E value | Species | Primers | Vector |
| 1 | pos-1/pie-1 | rk10c12.y1 | A | 116 2.0e−6 | M. javanica | posA | pCRII-TOPO |
| 2 | mei-1 | ra48f08.y1 | A | 293 1.0e−26 | M. incognita | meiA | pBKCMV |
| 3 | | ra33h10.y1 | B | 212 9.7e−18 | M. incognita | | pBKCMV |
| 4 | | ra69a10.y1 | C | 176 7.6e−14 | M. incognita | | pBKCMV |
| 5 | | ra38h05.y1 | D | 175 9.7e−14 | M. incognita | | pBKCMV |
| 6 | | ra30a04.y1 | E | 170 3.4e−13 | M. incognita | | pBKCMV |
| 7 | | ra71a06.y1 | F | 169 4.4e−13 | M. incognita | | Pbkcmv |
| 8 | | ra49c09.y1 | G | 169 4.7e−13 | M. incognita | | pBKCMV |
| 9 | | ra66a05.y1 | H | 83 0.0036 | M. incognita | | pBKCMV |
| 10 | rba-2 | ra38g07.y1 | A | 530 7.3e−52 | M. incognita | rbaA | pBKCMV |
| 11 | | ra20g12.y2 | B | 133 2.3e−08 | M. incognita | | pBKCMV |
| 12 (same as 11) | gpb-1 | ra20g12.y2 | A | 130 5.8e−8 | M. incognita | gpbA | pBKCMV |
| 13 | | ra64d09.y1 | B | 133 6.2e−08 | M. incognita | | pBKCMV |
| 14 (same as 10) | | ra38g07.y1 | C | 102 1.1e−05 | M. incognita | | pBKCMV |
| 15 | | ra55b12.y1 | D | 107 9.8e−05 | M. incognita | | pBKCMV |
| 16 | dif-1 | ra71g01.y1 | A | 166 3.7e−13 | M. incognita | difA | pBKCMV |
| 17 | | ra40e04.y1 | B | 164 5.8e−13 | M. incognita | | pBKCMV |
| 18 | | ra51d12.y1 | C | 129 1.1e−07 | M. incognita | | pBKCMV |
| | | | | | | | pBKCMV |
| 19 | skn-1 | ra53f10.y1 | A | 174 5.3e−17 | M. incognita | sknA | pBKCMV |
| 20 | plk-1 | ra03f10.y2 | A | 299 6.4e−27 | M. incognita | plkA | pBKCMV |
| 21 | | ra03f10.y1 | B | 277 1.5e−24 | M. incognita | | pBKCMV |
| 22 | | ra31c09.y1 | C | 234 7.7e−20 | M. incognita | | pBKCMV |
| 23 | | ra70c02.y1 | D | 207 6.0e−17 | M. incognita | | pBKCMV |
| 24 | | ra55c01.y1 | E | 169 8.4e−13 | M. incognita | | pBKCMV |
| 25 | | ra24d12.y1 | F | 165 2.1e−12 | M. incognita | | pBKCMV |
| 26 (same as 20) | par-1 | ra03f10.y2 | A | 341 5.1e−31 | M. incognita | | pBKCMV |
| 27 (same as 21) | | ra03f10.y1 | B | 320 8.9e−29 | M. incognita | | pBKCMV |
| 28 | | ra61c04.y1 | C | 186 4.6e−19 | M. incognita | | pBKCMV |
| 29 | | ra69h11.y1 | D | 167 4.9e−17 | M. incognita | | pBKCMV |
| 30 | | ra54f10.y1 | E | 167 5.0e−17 | M. incognita | | pBKCMV |
| 31 (same as 22) | | ra31c09.y1 | F | 206 1.6e−16 | M. incognita | | pBKCMV |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

All patent and scientific literature, computer programs and algorithms referenced is this specification are incorporated by reference herein in their entirety.

Bibliography

Altschul et al., Nucl. Acids Res. 25: 3389. 1997.

Arpagaus et al., J. Physiol. Paris 92:363-367, 1998.

Ausubel et al., “Current Protocols in Molecular Biology” John Wiley & Sons Inc, 1994-2001, Chapters 2 and 15.

Barker et al., Plant Mol. Biol. 2: 235-350, 1983.

Boxem et al., Development 126: 2227-2239, 1999.

Chuang, C. F. and Meyerowitz, E. M., Proc. Natl Acad. Sci. USA 97: 4985-4990, 2000.

Cully et al., Nature 371:_{—}707-711, 1994.

de Boer et al., Mol. Plant Microbe Interact. 12: 663-669, 1999.

Dent et al., EMBO J. 16: 5867-5879, 1997.

Ding et al., Mol. Plant Microbe Interact 11: 952-959, 1998.

Escobar et al., Molecular Plant-Microbe Interactions 12: 440-449, 1999.

Ferguson et al., Genes Dev. 10: 1543-1556, 1996.

Finney et al., Cell 55: 757-769, 1988.

Fire et al., Nature 391: 806-811, 1998.

Garfinkel et al., Cell 27: 143-153, 1983.

Greve, J. Mol. Appl. Genet. 1: 499-511, 1983.

Gumienny et al., Development 126: 1011-1022, 1999.

Hodgkin, Genetics 96: 649-664, 1980.

Hodgkin, Genetics 86: 275-287, 1977.

Hodgkin, Cell 8: 277-280, 1999.

Kamath et al., Genome Biology 2: 0002.1-0002.10, 2000.

Kennerdell, J. R. and Carthew, R. W., Cell 95: 1017-1026, 1998.

Kostrouchova et al., Development 125: 1617-1626, 1998.

Lilley et al., Mol. Biochem. Parasitol. 89: 195-207, 1997.

Maryon et al., J. Cell Sci. 111: 2885-2859, 1998.

Maryon et al., J. Cell. Biol. 134: 885-893, 1996.

McDaniel et al., Plant Journal 9: 55-61, 1996.

Opperman et al., Science 263: 221-223, 1994.

Piotte et al., Mol. Biochem. Parasitol. 99: 247-256, 1999.

Ray et al., Mol. Biochem. Parasitol. 83: 121-124, 1989.

Ray et al., Mol. Biochem. Parasitol. 68: 93-101, 1994.

Rosso et al., Mol. Plant Microbe Interact. 12: 585-591, 1999.

Salomon et al., EMBO J. 3: 141-146, 1984.

Schnabel et al., Curr. Opin. Genet. Deve. 1: 179-184, 1991.

Schnabel et al., Curr. Opin. Genet. Dev. 1: 179-184, 1991.

Smith et al., Nature 47: 319-320, 2000.

Tabara et al., Science 282: 430-431, 1998.

Tabara et al., Cell 99: 123-132, 1999.

Talesa et al., FEBS Letts. 357: 265-268, 1995.

The C. elegans Sequencing Consortium, Genome Sequence of the Nematode C. elegans, Science 282: 2012-2018, 1998.

Timmons & Fire, Nature 395: 854, 1998.

Timmons et al., Gene 263:103-112, 2001.

Vassilatis et al., J. Biol. Chem. 272: 33167-33174, 1997.

Wesley et al., Plant Journal 27: 581-590, 2001.

Wilson et al., Biochim. Biophys. Acta 1445: 99-109, 1999.

Xue et al., Science 261: 1324-1328, 1993.

Yochem et al., Development 126: 597-606, 1999.

Zarkower et al., Cell 70: 237-249, 1992.

What is claimed is:

1. A method for facilitating host resistance to at least one pathogen, said method including the step of generating a host which comprises one or more nucleotide sequences that is/are transcribable to one or more respective RNA molecules and that is/are substantially homologous and/or complementary to one or more respective nucleotide sequences of the genome of said at least one pathogen, such that upon exposure of said at least one pathogen to said host, there is down-regulation of expression of at least one of said respective nucleotide sequences of the genome of said at least one pathogen which thereby facilitates host resistance to said pathogen.

2. The method of claim 1, wherein said one or more nucleotide sequences are transcribed to one or more respective RNA molecules by one or more cells of said host.

3. The method of claim 2, wherein the one or more respective RNA molecules present in said cells of said host is ingested by said at least one pathogen, upon which there is down-regulation of expression of at least one of said respective nucleotide sequences of the genome of said at least one pathogen, which thereby facilitates host resistance to said at least one pathogen.

4. The method of claim 1, wherein said host comprises one or more substantially non-pathogenic microorganisms associated therewith, wherein said one or more nucleotide sequences are transcribed to one or more respective RNA molecules by said microorganism(s).

5. The method of claim 4, wherein said one or more microorganisms are ingested by said at least one pathogen, upon which there is down-regulation of expression of at least one of said respective nucleotide sequences of the genome of said at least one pathogen, which thereby facilitates host resistance to said at least one pathogen.

6. The method of claim 1, wherein there is down-regulation of expression of one or more RNA molecules transcribed from the or each said nucleotide sequence encoded by the genome(s) of said a least one pathogen.

7. The method of claim 1, wherein there is down-regulation of expression of a protein encoded by said one or more nucleotide sequence encoded by the genome of said at least one pathogen.

8. The method of claim 1, wherein the host is a plant.

9. The method of claim 1, wherein the plant is a tobacco plant.

10. The method of claim 8, wherein the plant is a monocotyledonous plant.

11. The method of claim 10, wherein the monocotyledonous plant is a pineapple plant.

12. The method of claim 1, wherein said at least one pathogen is an endoparasitic nematode of the family Heteroderidae.

13. The method of claim 12, wherein the nematode is of the genus Meloidogyne.

14. The method of claim 1, wherein said one or more nucleotide sequences are a plurality of nucleotide sequences.

15. The method of claim 14, wherein each of said plurality of nucleotide sequences is from a respective said at least one pathogen.

16. The method of claim 14, wherein said plurality of nucleotide sequences is from a single pathogen.

17. A genetically-modified host produced according to the method of claim 1.

18. The genetically-modified host of claim 17, which is a plant.

19. The genetically-modified host of claim 18, which is a tobacco plant.

20. The genetically-modified host of claim 18, which is a monocotyledonous plant.

21. The genetically-modified host of claim 20, wherein the monocotyledonous plant is a pineapple plant.

22. The genetically-modified host of claim 18, which is nematode resistant.

23. A genetic construct for facilitating host resistance to a pathogen, said genetic construct comprising one or more nucleotide sequences that is/are transcribable to an RNA molecule and that is/are substantially homologous and/or complementary to one or more nucleotide sequences encoded by the genome of said pathogen, such that upon exposure of said pathogen to one or more RNA molecules transcribed from said one or more nucleotide sequences, there is down-regulation of expression of at least one of said respective nucleotide sequences of the genome of said pathogen.

24. A genetic construct for facilitating host resistance to a pathogen, said genetic construct comprising one or more nucleotide sequences selected from the group consisting of:
(i) a nucleotide sequence transcribable to an RNA molecule comprising an RNA sequence that is/are substantially homologous to an RNA sequence encoded by the genome of said pathogen;
(ii) a reverse complement of the nucleotide sequence of (i);
(iii) a combination of the nucleotide sequences of (i) and (ii);
(iv) multiple copies of the nucleotide sequences of (i), (ii) or (iii), optionally separated by a spacer sequence;
(v) a combination of the nucleotide sequences of (i) and (ii), wherein the nucleotide sequence of (ii) represents an inverted repeat of the nucleotide sequence of (i), separated by a spacer sequence; and
(vi) a combination as described in (v), wherein the spacer sequence comprises an intron sequence sliceable from said combination;
such that upon exposure of said pathogen to one or more RNA molecules transcribed from said one or more nucleotide sequences, there is down-regulation of expression of at least one said nucleotide sequence of the genome of said pathogen.

25. The genetic construct of claim 23 or claim 24, wherein said one or more nucleotide sequences are operably linked to a promoter and comprising one or more transcription enhancing sequences.

26. The genetic construct of claim 25, wherein said one or more nucleotide sequences are selected from the group consisting of those set forth in SEQ ID NO:1; SEQ ID NO:2; SEQ ID NO:3; SEQ ID NO:4; SEQ ID NO:5; SEQ ID NO:6; SEQ ID NO:7 and SEQ ID NO:8..

27. The genetic construct of claim 25, wherein said one or more nucleotide sequences are selected from the group consisting of those set forth in SEQ ID NO:1; SEQ ID NO:2 and SEQ ID NO:3.

28. The genetic construct of claim 25, wherein said genetic construct is pUQC477 or pUQC136.

29. A method for facilitating pineapple plant resistance to nematode infection and/or infestation, said method including the step of generating a pineapple plant which comprises one or more nucleotide sequences that is/are transcribable to one or more respective RNA molecules and that is/are substantially homologous and/or complementary to one or more respective nucleotide sequences of the genome of said nematode, such that upon exposure of said nematode to said pineapple plant comprising said one or more respective RNA molecule(s), there is down-regulation of expression of at least one of said respective nucleotide sequences of the genome of said nematode which thereby facilitates nematode resistance of said pineapple plant.
